(19) United States
(12) Patent Application Publication
(10) Pub. No.: US 2025/0072888 A1
(43) Pub. Date: Mar. 6, 2025

(54) SUTURING DEVICES AND RELATED SYSTEMS AND METHODS

(71) Applicant: Intuitive Surgical Operations, Inc., Sunnyvale, CA (US)

(72) Inventor: Ryan Abbott, Sunnyvale, CA (US)

(21) Appl. No.: 18/821,220

(22) Filed: Aug. 30, 2024

Related U.S. Application Data
(60) Provisional application No. 63/536,350, filed on Sep. 1, 2023.

(51) Int. Cl. A61B 17/04; A61B 17/00; A61B 17/06; A61B 34/37

(52) U.S. Cl. CPC A61B 17/0491; A61B 17/0469; A61B 17/06066; A61B 2017/00367; A61B 2017/00477; A61B 2017/047; A61B 34/37

(57) ABSTRACT

Implementations of a suturing device and related methods are provided. The suturing device includes a housing that defines an arcuate needle track having a first end and a second end and a gap between the first end and second end of the arcuate needle track. The suturing device further includes a reciprocating drive to advance an arcuate needle along the arcuate needle track that includes a drive pawl to drive the arcuate needle along the arcuate needle track.

### CROSS-REFERENCE TO RELATED APPLICATION

The present patent application claims the benefit of priority to U.S. Provisional Patent Application No. 63/536,350, filed Sep. 1, 2023. The aforementioned patent application is incorporated by reference herein in its entirety for all purposes.

### TECHNICAL FIELD

Aspects of the present disclosure relate to devices, systems, and methods for performing a suturing procedure. For example, aspects of the present disclosure relate to suturing devices in support of remote surgical, diagnostic, therapeutic, and other treatment procedures. Further aspects of the disclosure relate to methods of operating such devices.

### INTRODUCTION

Sutures are used in a variety of surgical and other applications, such as closing ruptured or incised tissue, soft tissue attachment, attachment of grafts, etc. Additionally, sutures may have other medical and/or non-medical uses. Conventionally, suturing is accomplished by penetrating tissue with the sharpened tip of a arcuate needle that has a thread of suturing material attached to the opposite blunt end of the needle. The needle is then pulled through the tissue, causing the attached thread of suturing material to follow the path of the needle. Typically, a knot is tied at the trailing end of the thread to anchor the first stitch. This action is performed repetitively with application of tension to the needle to pull a length of the thread through the tissue using subsequent stitches until the tissue is sutured as desired with one or more stitches.

While the above-described suturing process can be performed manually, automated suturing systems also exist. Such systems can include a needle driver device that has an open, C-shaped portion into which tissue segments are introduced. The C-shaped portion defines two arms, each with an entry/exit point for a curved needle. The curved needle is driven around a track (generally following the C-shaped portion) and across the opening in the C-shaped portion to draw a thread of suturing material into the needle driver device through the tissue segments, similar to the manual suturing process discussed above. It is desirable to provide needle driver devices that occupy a minimal amount of space relative to a size (e.g., gauge and/or radius) of the needle and that have a lower relative number of mechanical components. The present disclosure addresses these, and other issues in the art, as set forth below.

### SUMMARY

Example embodiments of the present disclosure may solve one or more of the above-mentioned problems and/or may demonstrate one or more of the above-mentioned desirable features. Other features and/or advantages may become apparent from the description that follows.

In accordance with some aspects of the present disclosure, a suturing device is provided. The suturing device includes a housing that defines an arcuate needle track. The arcuate needle track has a first end and a second end. The housing further defines a gap between the first end and second end of the arcuate needle track. The gap is configured to receive tissue therein to be sutured. The device further includes a drive to advance an arcuate needle about the arcuate needle track and across the gap. The drive includes at least one drive pawl operably coupled to an actuator. The at least one drive pawl is configured to engage and disengage from the arcuate needle. The drive is configured to advance the at least one drive pawl along a drive pawl pathway from a starting position to an ending position during a drive stroke. The drive pawl pathway may be linear when the at least one drive pawl is not engaged with the arcuate needle.

In accordance with some implementations, the drive pawl pathway can be linear or curved when the at least one drive pawl is engaged with and advancing the arcuate needle. In some implementations, the drive pawl pathway can form a tangent with respect to a needle pathway traversed by the arcuate needle. If desired, the drive pawl pathway can be laterally offset with respect to a central longitudinal axis of the suturing device.

In some implementations, the at least one drive pawl can include a first drive pawl and a second drive pawl, wherein the first drive pawl engages the arcuate needle proximate a first lateral side of the arcuate needle track, and the second drive pawl engages the arcuate needle proximate a second lateral side of the arcuate needle track. If desired, one or both of the first drive pawl and second drive pawl can traverse a linear drive pawl pathway from the starting position to the ending position. In some implementations, the first drive pawl and second drive pawl can move in tandem when the actuator is actuated. The first drive pawl and second drive pawl can be mechanically coupled or mechanically decoupled. The drive can further include a yoke that in turn defines a first distally extending arm and a second distally extending arm. The first drive pawl can be operably coupled to the first distally extending arm and the second drive pawl can be operably coupled to the second distally extending arm. The first drive pawl can be antipodally located with respect to the second drive pawl, or offset from an antipodal arrangement, as desired.

In some implementations, the first drive pawl and second drive pawl can physically contact the arcuate needle simultaneously when the arcuate needle is in a predetermined position within the arcuate needle track. In some implementations, the first drive pawl can be actuated independently of the second drive pawl.

In accordance with further aspects of the disclosure, the first drive pawl can be configured to advance the arcuate needle along a first rotational direction along the arcuate needle track when the first drive pawl is advanced along a distal direction. If desired, the second drive pawl can be configured to advance the arcuate needle along a second rotational direction opposite the first rotational direction along the arcuate needle track when the second drive pawl is advanced along a distal direction. In some implementations, the first drive pawl can be configured to advance the arcuate needle along the second rotational direction along the arcuate needle track when the second drive pawl is retracted along a proximal direction. In some implementations, the second drive pawl can be configured to advance the arcuate needle along the first rotational direction along the arcuate needle track when the second drive pawl is retracted along a proximal direction. In further implementations, the first drive pawl can be configured to advance the arcuate needle along a first rotational direction along the arcuate needle track when the first drive pawl is advanced along a distal direction, and the second drive pawl can be configured to advance the arcuate needle along the first rotational direction along the arcuate needle track when the second drive pawl is retracted along a proximal direction.

In accordance with further aspects of the disclosure, the housing can define a longitudinal passage therethrough, and the suturing device can further include an elongate tool slidably disposed in the longitudinal passage. If desired, the longitudinal passage can be formed along a direction that intersects the arcuate needle track, or along a direction that does not intersect the arcuate needle track.

In some implementations, a distal end region of the elongate tool can define at least one needle engagement surface to grasp a tip portion of the arcuate needle. For example, the drive can be configured to advance the needle along a forward direction along the arcuate needle track wherein the tip portion of the arcuate needle is located at a leading end of the needle. Moreover, the drive can be configured to retract the arcuate needle along a rearward direction along the arcuate needle track that is opposite to the forward direction when the a portion of the arcuate needle is grasped by the elongate tool to separate the tip portion of the arcuate needle from a main body portion of the arcuate needle.

In some implementations, the suturing device can further include a length of suture material having a distal end coupled to the tip portion of the arcuate needle. The length of suture material can extend proximally past the at least one needle engagement surface of the elongate tool and through a cinch operably coupled to the at least one needle engagement surface of the elongate tool to form a suture loop. If desired, a proximal end of the suture can be tensioned to shorten the suture loop. In accordance with some aspects of the disclosure, the at least one needle engagement surface of the elongate tool and the cinch can be configured to be detached from the elongate tool and from the suturing device as an integral unit to permit the remainder of the elongate tool to be withdrawn proximally through the longitudinal passage.

In accordance with further aspects of the present disclosure, the suturing device can further include an elongate tool slidably disposed in the longitudinal passage, wherein a distal end region of the elongate tool defines at least one tissue engagement surface to engage tissue of a patient. In some implementations, the suturing device can further include an endoscope operably coupled with the housing. If desired, the endoscope can be removably coupled to the housing. The drive can be configured to advance the arcuate needle about the arcuate needle track along an angular extent of at least 360 degrees in a plurality of drive strokes. If desired, the at least one drive pawl can be configured to engage a surface formed into an outer radial face of the arcuate needle.

In further accordance with the disclosure, the housing of the suturing device can define a proximally extending passageway along a longitudinal axis of the drive, wherein the passageway is configured to receive an elongate tool comprising a tissue capture portion comprising a helix member. The tissue capture portion can be removed from the passageway. The passageway can be configured to further receive an elongate tool supporting a replacement tip portion for coupling to the arcuate needle after removal of the tissue capture portion from the passageway.

In some implementations, the drive can be configured to advance the drive pawl from the starting position along a linear path prior to engagement with the arcuate needle. If desired, the starting position of the drive pawl can be located proximally with respect to a center point of the arcuate needle track. In some implementations, the drive can be configured to advance the drive pawl along a linear path after disengagement with the arcuate needle until the drive pawl reaches the ending position. If desired, the ending position of the drive pawl can be located distally with respect to a center point of the arcuate needle track.

In still further accordance with the disclosure, a suturing device is provided including a housing. The housing, defines a gap between a first housing portion and a second housing portion, wherein the gap is configured to receive tissue therein to be sutured. The suturing device further includes a drive to advance an arcuate needle across the gap during a drive stroke. The arcuate needle can have a length of suturing material extending proximally therefrom. The suturing device further includes an elongate tool having a detachable coupling at a distal end of the elongate tool. The detachable coupling can define therein at least one needle engagement surface to engage with a tip portion of the arcuate needle. The detachable coupling can further define a cinch to engage a proximal portion of the length of suturing material. Engagement of the arcuate needle with the detachable coupling can form a loop of suturing material that can be shortened in length by advancing the length of suturing material along a proximal direction through the cinch. In some implementations, the housing can include an arcuate needle track having a first end and a second end, wherein the gap is defined between the first end and second end of the arcuate needle track.

In further accordance with the disclosure a suturing device is provided that includes a housing. The housing can define an arcuate needle track having a first end and a second end, wherein the housing further defines a gap between the first end and second end of the arcuate needle track to receive tissue therein to be sutured. The housing can further define a proximally extending passageway along a direction that intersects the arcuate needle track. The device can further include a drive to advance an arcuate needle about the arcuate needle track and across the gap. The drive can include at least one drive pawl operably coupled to an actuator. The actuator can be slidably received in the proximally extending passageway and extending proximally out of the housing. The suturing device can further include an elongate tool being slidably disposed within the proximally extending passageway alongside the actuator. The elongate tool can include an instrumentality at a distal end thereof to grasp at least one of the arcuate needle and tissue of a patient.

In accordance with further aspects of the disclosure, the elongate tool can include a tissue capture portion comprising a helix member. If desired, the tissue capture portion can be removed from the passageway, and the passageway can be configured to receive an elongate tool supporting a replacement tip portion for coupling to the arcuate needle.

The disclosure further provides implementations of a suturing device including a housing. The housing defines an arcuate needle track. The arcuate needle track has a first end and a second end, and the housing further defines a gap between the first end and second end of the arcuate needle track. The gap is configured to receive a tissue therein to be sutured. The device further includes a drive to advance an arcuate needle about the arcuate needle track and across the gap. The drive includes a first drive pawl and a second drive pawl. The first drive pawl can be configured to be advanced by the drive along a first direction into contact with the arcuate needle to advance the arcuate needle in a forward direction along the needle track by a first distance. The second drive pawl can be configured to be retracted by the drive along the first direction into contact with the arcuate needle to continue to advance the arcuate needle in the forward direction along the needle track by a second distance.

In accordance with further aspects, the first drive pawl can engage the arcuate needle proximate a first lateral side of the arcuate needle track, and the second drive pawl can engage the arcuate needle proximate a second lateral side of the arcuate needle track. The first drive pawl can be configured to advance the arcuate needle along a first rotational direction along the arcuate needle track when the first drive pawl is advanced along a distal direction, and the second drive pawl can be configured to advance the arcuate needle along the first rotational direction along the arcuate needle track when the second drive pawl is retracted along a proximal direction.

The disclosure further includes methods of operating a suturing device, and methods of conducting a suturing procedure. In accordance with some implementations, a method of operating a suturing device is provided. The method includes providing a suturing device that includes a housing, and the housing defining an arcuate needle track having a first end and a second end. The housing defines a gap in the housing between the first end and second end of the arcuate needle track. The suturing device further includes a drive to advance an arcuate needle about the arcuate needle track and across the gap. The method further includes advancing a first drive pawl along a first direction into contact with the arcuate needle to advance the arcuate needle in a forward direction along the needle track by a first distance. The method still further includes advancing a second drive pawl along a second direction opposite to the first direction into contact with the arcuate needle to continue to advance the arcuate needle in the forward direction along the needle track by a second distance.

In accordance with some implementations, the first drive pawl can engage the arcuate needle on a first side of the gap to advance the needle in the forward direction, and the second drive pawl can engage the arcuate needle on a second side of the gap to continue advance the needle in the forward direction. The first drive pawl can be advanced in the first direction by actuating a first actuator, and the second drive pawl can be advanced in the second direction by actuating a second actuator that is separably actuatable from the first actuator. In some implementations, the first drive pawl can be advanced in the first direction by actuating a first actuator along the first direction, and the second drive pawl can be advanced in the second direction by actuating the first actuator along the second direction. In some embodiments, the first pawl and the second pawl can each be advanced along a linear path that is parallel to a longitudinal axis of the housing.

In some implementations, the method can further include advancing a tissue capture catheter distally through a passageway formed in the housing to engage tissue of a patient, pulling the tissue of the patient into the gap of the housing, and advancing the arcuate needle through the tissue of the patient. In still further implementations, the method can further include advancing a needle capture catheter distally through the passageway formed in the housing, capturing a tip portion of the needle in a suture cinch detachably coupled to a distal end of the needle capture catheter, and advancing the second drive pawl along the first direction to advance the needle along a rearward direction opposite to the forward direction to detach the tip portion of the needle from a main body portion of the needle. In some implementations, a length of suturing material can extend from the tip portion of the needle and into the suture cinch to form a loop of suturing material.

In accordance with yet further aspects of the disclosure, the method can further include applying tension to the suturing material to shorten the loop of suturing material to compress the tissue of the patient. If desired, the method can further include detaching the cinch from the distal end of the needle capture catheter to release the needle tip that is joined to the cinch into the patient.

In accordance with still further aspects of the disclosure, the method can further include withdrawing the needle capture catheter proximally through the passageway formed in the housing, and advancing a second needle capture catheter into the passageway formed in the housing. The second needle capture catheter can include a cinch removably coupled to a distal end thereof, a new needle tip coupled to a distal end portion of the cinch, and a length of suture material coupled to the new needle tip that is routed through the cinch and the passageway formed in the housing. The method can still further include aligning the new needle tip with the arcuate needle track, and advancing the second drive pawl along the second direction to advance the main portion of the needle along the forward direction to join the new needle tip to the main body portion of the needle.

The disclosure further provides an arcuate needle having a main body portion. The main body portion is defined by a leading end, a trailing end, and an arcuately shaped body. The arcuately shaped body defining a bore along its length from the leading end to the trailing end. The bore extends radially inwardly through an inner radial wall of the arcuately shaped body. The main body portion has a “C” shaped cross section along its length.

In some implementations, the arcuate needle can further include a needle tip portion removably coupled to the leading end of the main body portion. The needle tip portion can include a pointed distal end and a proximal end terminating in a proximal coupling to couple to the main body portion. The proximal coupling of the needle tip portion can include a boss configured to be received by a cavity defined in the leading end of the main body portion of the needle. The arcuate needle can include a piece of suturing material that extend proximally from the boss of the needle tip portion into the bore of the main body portion.

In accordance with further aspects of the disclosure, an outer radial surface of the main body portion of the arcuate needle can define at least one indentation therein. The at least one indentation can be defined by at least one facet laying in a plane that is transverse to a central arcuate axis of the main body portion of the needle. The at least one indentation can include a plurality of indentations. Each indentation can be formed by two intersecting facets. At least one of said intersecting facets can lay in a plane that is oblique to a central arcuate axis of the main body portion of the needle. If desired, at least one of the indentations can include a lower surface that defines a peak along a bottom of the indentation. In some implementations, at least two of the indentations can include portions that are located antipodally.

Additional objects, features, and/or advantages will be set forth in part in the description which follows, and in part will be obvious from the description, or may be learned by practice of the present disclosure and/or claims. At least some of these objects and advantages may be realized and attained by the elements and combinations particularly pointed out in the appended claims.

It is to be understood that both the foregoing general description and the following detailed description are illustrative and explanatory only and are not restrictive of the claims; rather the claims should be entitled to their full breadth of scope, including equivalents.

### BRIEF DESCRIPTION OF THE DRAWINGS

The present disclosure can be understood from the following detailed description, either alone or together with the accompanying drawings. The drawings are included to provide a further understanding of the present disclosure and are incorporated in and constitute a part of this specification. The drawings illustrate one or more embodiments of the present teachings and together with the description explain certain principles and operation. In the drawings,

FIG. 1 depicts an isometric left side view of a distal end region of a suturing device in accordance with some embodiments of the present disclosure.

FIG. 2 depicts a wireframe view of the device of FIG. 1 showing the locations of interior structures.

FIG. 3 is an isometric right side wireframe view of the device of FIG. 1 showing the locations of interior structures.

FIG. 4 is a vertical section view taken down an axial centerline of the device of FIG. 1 illustrating relative placement of interior components.

FIG. 5 is an enlarged view of FIG. 4.

FIG. 6 is a horizontal section view taken down an axial centerline of the device of FIG. 1 illustrating relative placement of interior components.

FIG. 7 is an upper left side isometric exploded view of the device of FIG. 1.

FIG. 8 is a lower right side isometric exploded view of the device of FIG. 1.

FIG. 9 is an upper left side isometric view of a housing component of the device of FIG. 1.

FIG. 10 is a lower left side isometric view of a housing component of the device of FIG. 1.

FIG. 11 is an upper distal end isometric view of a housing component of the device of FIG. 1.

FIG. 12 is a left upper isometric view of a needle drive component of the device of FIG. 1

FIG. 13 is a right upper proximal isometric view of a needle drive component of the device of FIG. 1

FIG. 14 is an upper left side isometric view of a distal end portion of a needle tip delivery catheter in accordance with the present disclosure.

FIG. 15 is a distal end view of the component of FIG. 14.

FIG. 16 is a proximal end view of the component of FIG. 14.

FIG. 17 is a top plan view of the component of FIG. 14.

FIG. 18 is a proximal isometric cutaway view along the line “FIGS. 18, 19” in FIG. 17.

FIG. 19 is a distal isometric cutaway view along the line indicated in FIG. 17.

FIG. 20 is a proximal isometric cutaway view along the line indicated in FIG. 17.

FIG. 21 is a left side plan view of the component of FIG. 14.

FIG. 22 is an isometric cutaway view of the component of FIG. 14 along line “FIG. 22” in FIG. 21.

FIG. 23 is an isometric view of a proximal portion of the needle delivery catheter of FIG. 1.

FIG. 24 is a bottom plan view of the component of FIG. 23.

FIG. 25 is a cutaway isometric view of the component of FIG. 23 adjacent the component of FIG. 14 illustrating a tapered cavity of the component of FIG. 14 configured to receive a distal end portion of the component of FIG. 25 along line “FIG. 27” in FIG. 26.

FIG. 26 is a left side isometric view of a distal end portion of a tissue capture catheter component of the device of FIG. 1.

FIG. 27 is a right-side isometric view of a distal end portion of a tissue capture catheter component of the device of FIG. 1.

FIG. 28 is an upper left isometric view of a main body portion of a needle for use in the device of FIG. 1.

FIG. 29 is an upper proximal isometric view of the component of FIG. 28.

FIG. 30 is an upper distal isometric view of the component of FIG. 28.

FIG. 31 is an upper isometric cutaway view of the component of FIG. 28 along a central horizontal plane.

FIG. 32 is a top plan view of the component of FIG. 28.

FIG. 33 is a bottom plan view of the component of FIG. 28.

FIGS. 34-44 illustrate steps of operation of the device of FIG. 1 to advance an arcuate needle fully about a needle track of the device.

FIGS. 45-50 illustrate the steps of operation of the device of FIG. 1 to remove a needle tip from a main body portion of the needle.

FIGS. 51-52 illustrate the steps of operation of the device of FIG. 1 to remove a needle tip from a main body portion of the needle.

FIG. 53 depicts a schematic view of a suturing device in accordance with some embodiments of the present disclosure in combination with an endoscope and actuator to facilitate suturing, or aspects thereof.

FIG. 54 is a schematic diagram for a robotically-assisted manipulator system, according to some examples.

FIG. 55A is a schematic diagram of an instrument system according to examples described herein.

FIG. 55B illustrates a distal portion of the instrument system of FIG. 55A with an extended example of an instrument according to examples described herein.

### DETAILED DESCRIPTION

The present disclosure provides various embodiments of suturing devices, systems, and methods. Suturing devices according to various embodiments of the present disclosure include features that facilitate suturing operations, particularly in endoscopic procedures.

In accordance with some aspects of the present disclosure, a suturing device is provided. The suturing device includes a housing that defines an arcuate needle track.

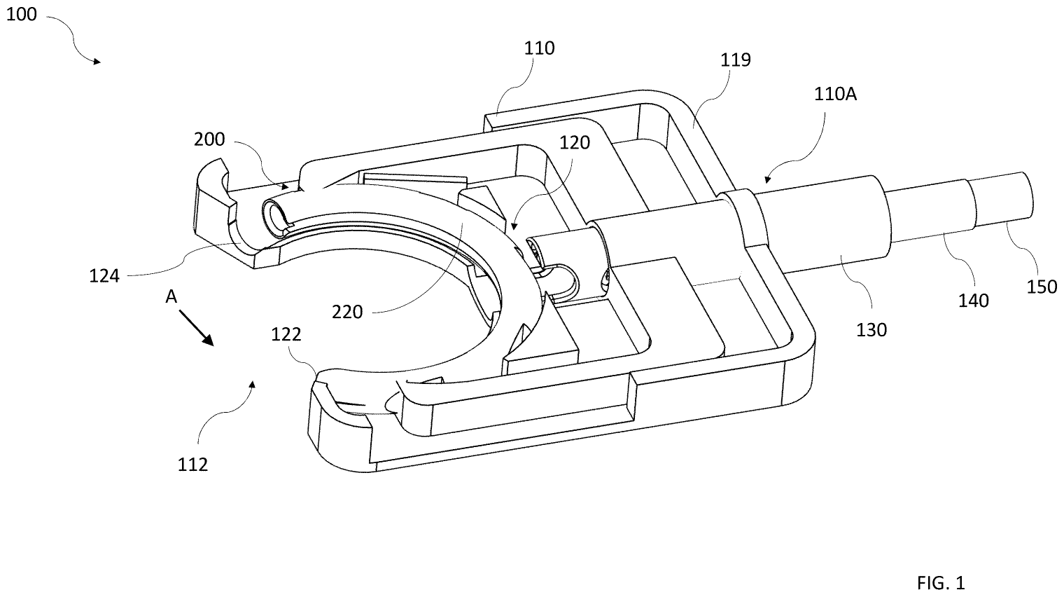

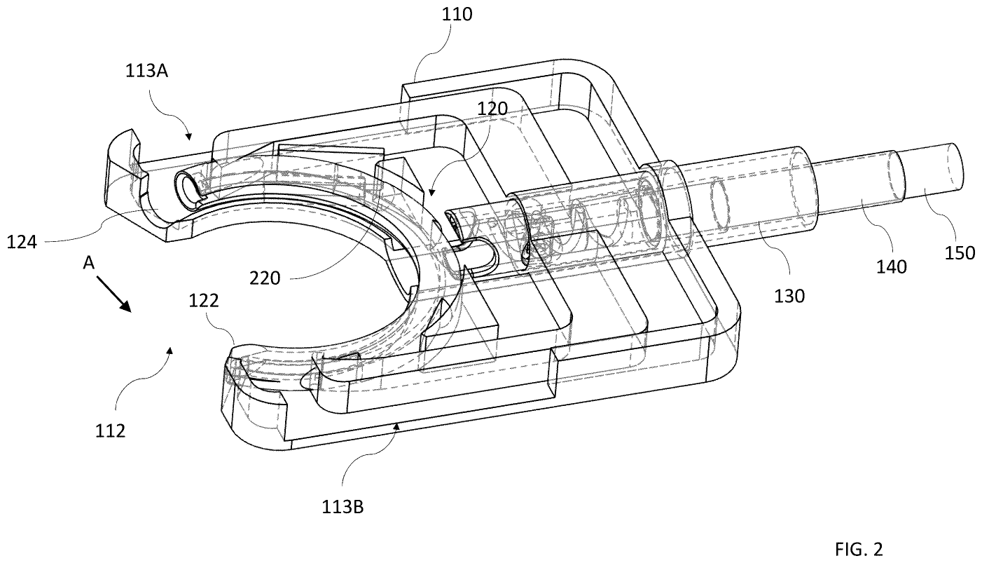

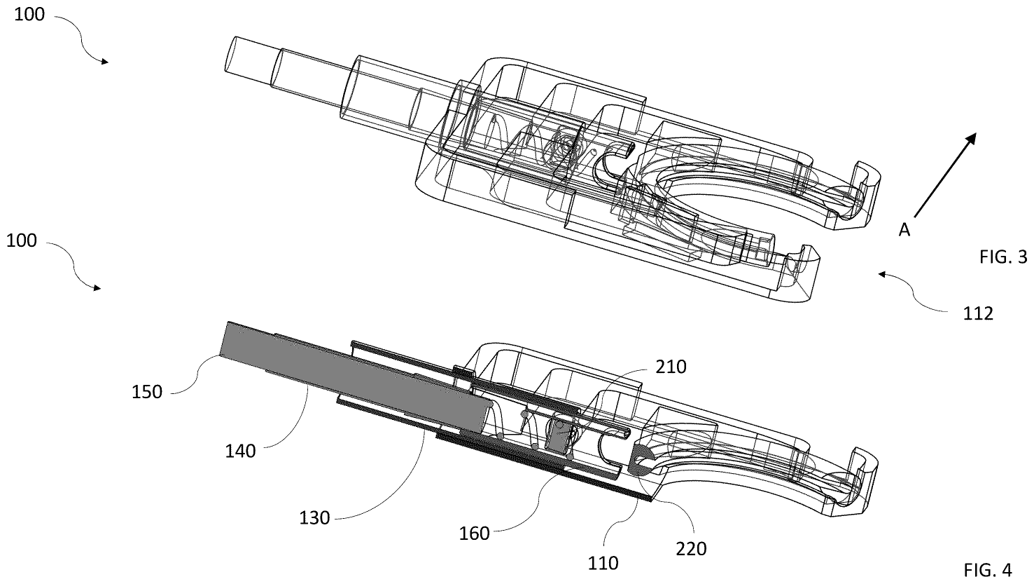

For purposes of illustration, and not limitation, a representative implementation of a suturing device is presented in FIGS. 1-8.

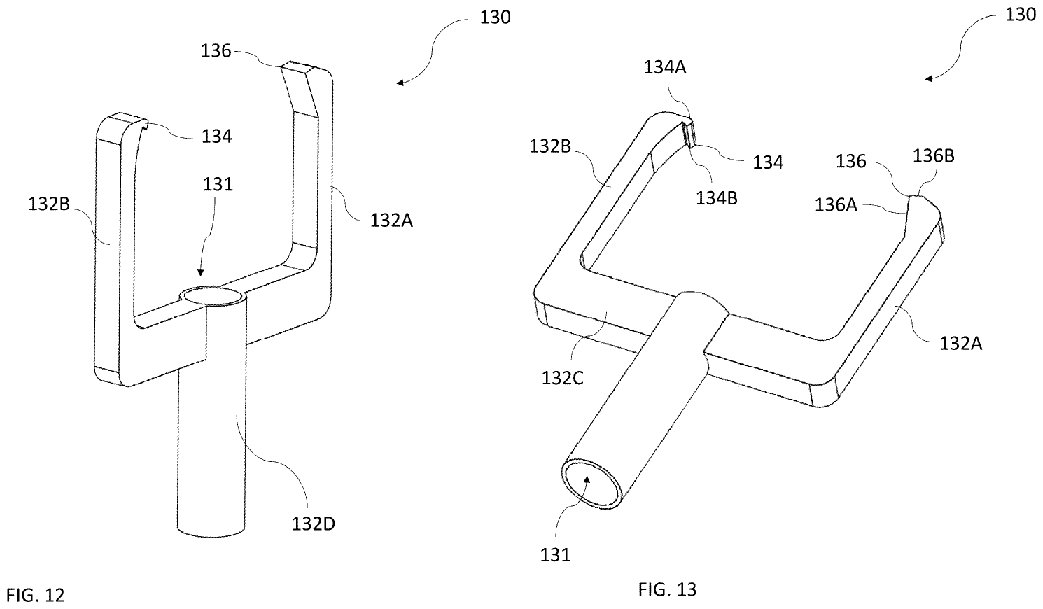

In accordance with some aspects of the present disclosure, a suturing device 100 is provided. The suturing device includes a housing 110 that defines an arcuate needle track 120 therein. The arcuate needle track 120 has a first end 122, wherein a needle 200 enters the track 120, and a second end 124 where the needle exits the track and crosses a gap 112 defined between the first end 122 and second end 124 of the arcuate needle track 120. The gap 112 is configured to receive tissue therein to be sutured. The device 100 further includes a drive (130, discussed in further detail below) to advance an arcuate needle (e.g., 200) about the arcuate needle track 120 and across the gap 112. As depicted, the drive 130 is slidably disposed within a cavity defined within the housing and may move between a distal most position and a proximal most position. A hollow tubular portion 132D of the drive 130 (FIG. 12) is received within a bore 110A formed in a proximal end wall 119 of the housing 110. A distal portion of the drive 130 includes arms or arms 132A, 132B having respective pawls 134, 136 (FIGS. 12-13) to releasably engage with the needle 200 to drive the needle around the arcuate needle track 120.

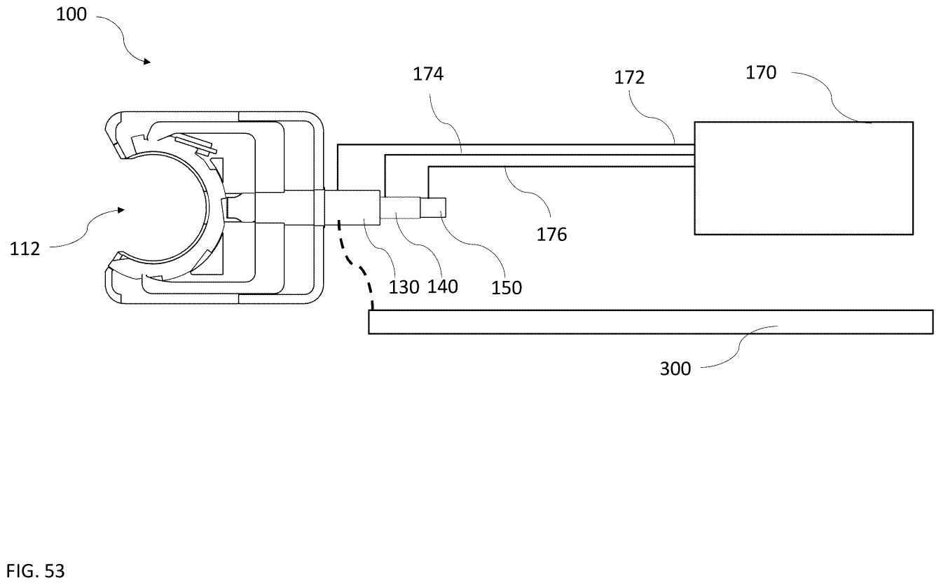

Not pictured but forming a part of the suturing device include proximal portions of the device that are operably coupled to an actuator system 170 to actuate each portion of the device 100 (see FIG. 53). The suturing device may be used alone, or may be permanently or removably coupled to an endoscope, such as a standard endoscope 300 (FIG. 53).

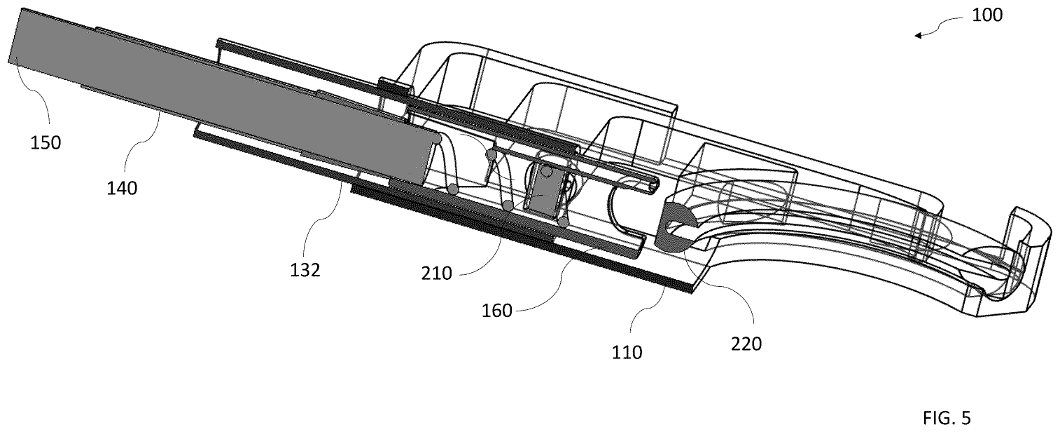

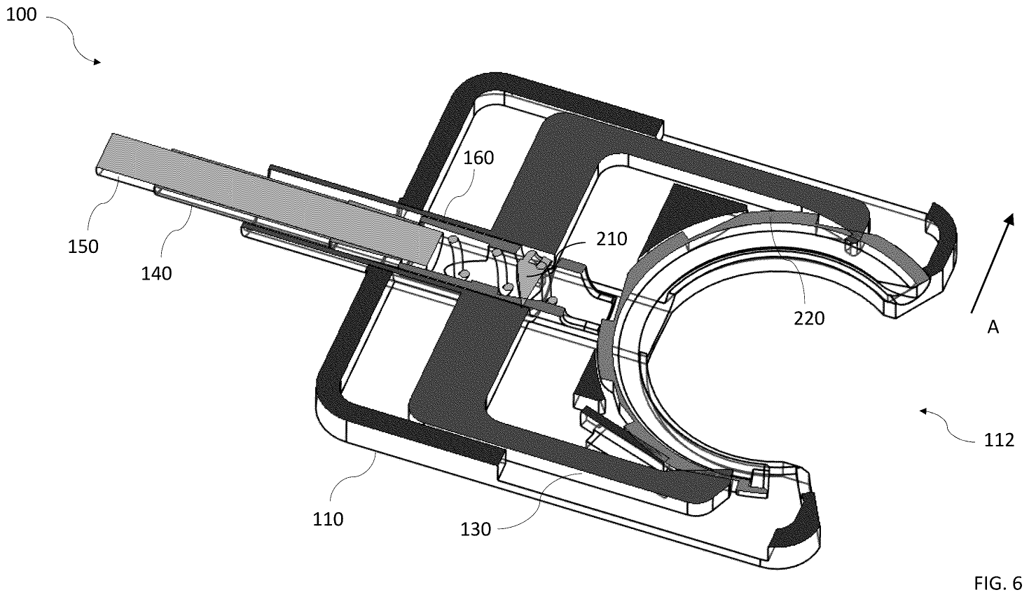

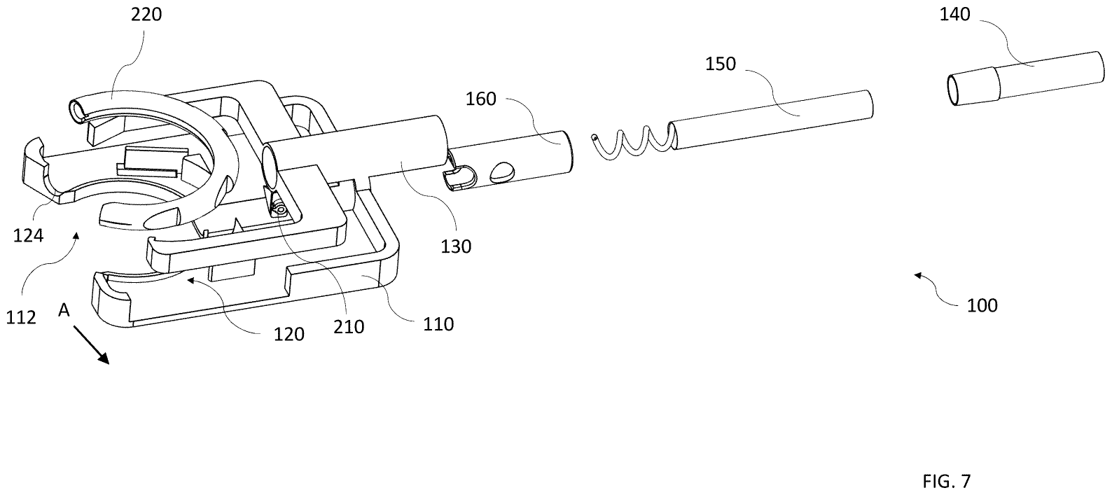

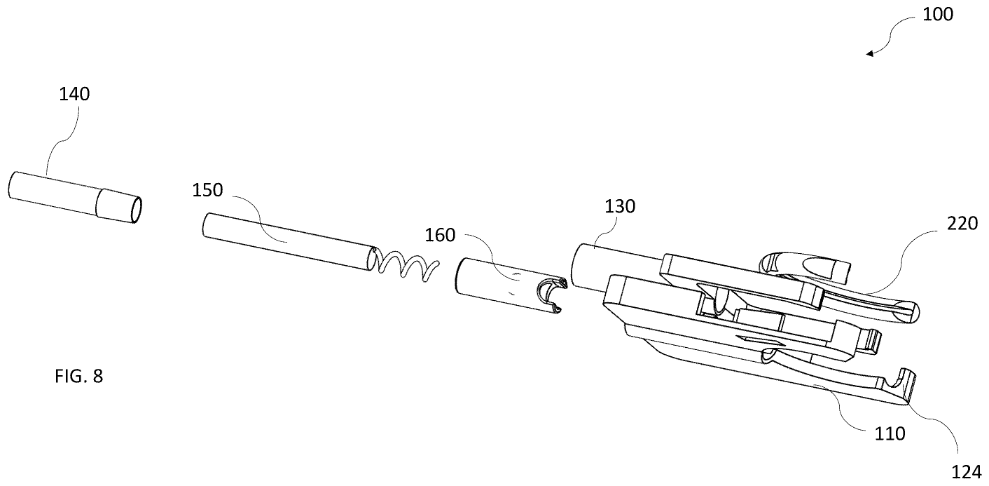

FIG. 5 is a vertical section view taken down an axial centerline of the device of FIG. 1 illustrating relative placement of interior components of device 100. FIG. 6 is a horizontal section view taken down an axial centerline of the device of FIG. 1 illustrating relative placement of interior components. The drive 130 is located radially outwardly of other components of the system. Located radially inwardly of the proximally extending tubular portion 132D of the drive 130 is a distal end portion 160 of a needle tip delivery catheter that serves to deliver a new needle tip 210 to install the needle tip 210 on a main body portion 220 of arcuate needle 200. Distal end portion 160 is slidably disposed within a bore of tubular portion 132D. Distal end portion 160 of needle tip delivery catheter removably couples to a proximal portion 140 of catheter, discussed in detail below. Located radially inwardly of the other system components is a tissue capture catheter 150 that is slidably disposed within a bore of section 140, discussed in further detail below. FIG. 7 is an upper left side isometric exploded view of the device 100 of FIG. 1, whereas FIG. 8 is a lower right side isometric exploded view of the device 100 of FIG. 1 to more clearly illustrate each component of device 100, discussed in further detail below.

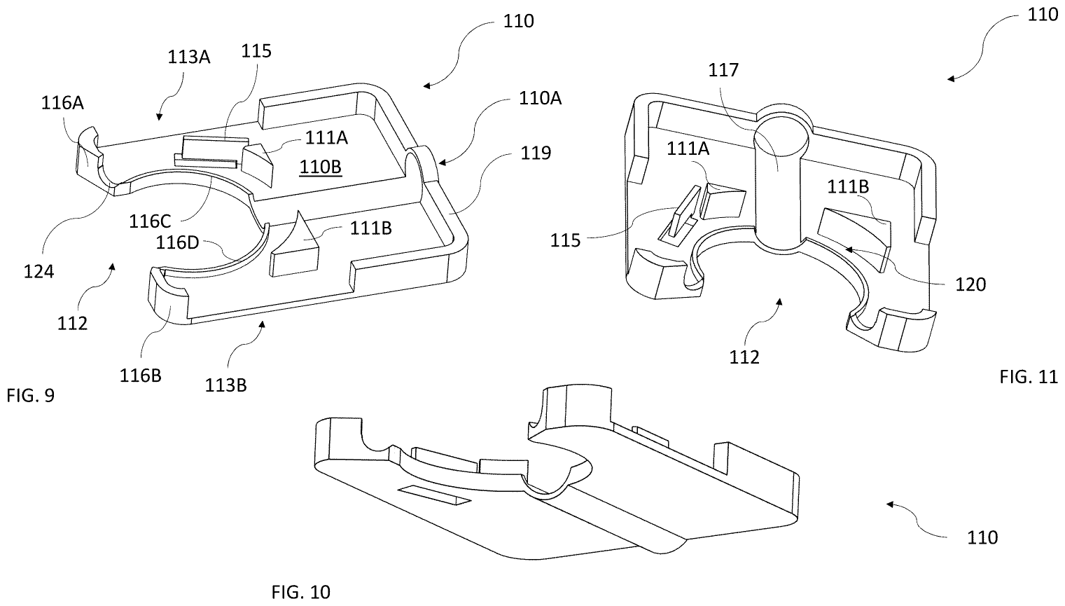

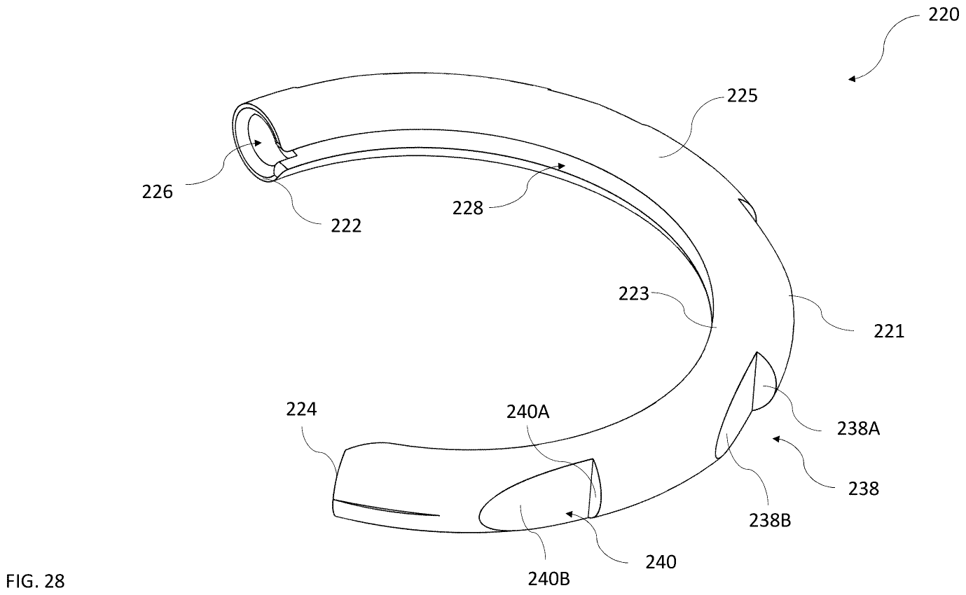

FIGS. 9-11 illustrate various views of the housing 110 of the device 100, which also includes a cover portion (not shown). As illustrated, housing 110 is rectilinear in shape, but may have any suitable shape, as desired. Housing 110 includes a floor 110B that can be substantially planar, which can include one or more upwardly extending walls to define a cavity therein to house system components. A proximal end wall 119 traverses a full width of the housing 110 along a proximal end, and defines a bore 110A therethrough for receiving other components of the device 100. A distal end wall is formed from two distal most portions 116A, 116B at a distal end of the housing 110 on either side of gap 112. Third and fourth distal portions or wall sections 116C, 116D of the distal wall are arcuate and at least partially surround the gap 112, wherein the gap is generally arcuately shaped. Needle track 120 is formed in part by wall sections 116C, 116D in cooperation with bosses or guides 111A, 111B. Each boss 111A, 111B includes a distally facing surface that is arcuately shaped when viewed in a top view. The cover portion of the housing (not shown) can similarly be provided with walls that correspond to wall sections 116C, 116D to permit an arcuate groove to be formed around the full extent of gap 112. Each lateral side of the housing 110 defines an elongate window 113A, 113B therethrough to permit arms 132A, 132B of the drive 130 to deflect laterally outwardly when pawls 134, 136 pass over a radially outward surface 221 of the main body portion 220 of the needle 200 (FIG. 28) or past the laterally outward face of deflectable pawl 115. Pawl 115 is integrally formed with housing 110 in some embodiments, but may be a separate component. Pawl 115 prevents undesired backward motion of the needle, 200, discussed in further detail below. If desired, the floor 110B of housing 110 and/or the cover portion (not shown) of the housing 110 may define a distally extending longitudinal channel along its length to the gap 112 to slidably receive the needle drive 130, discussed below.

FIG. 12 is a left upper isometric view of needle drive 130 component of device 100, and FIG. 13 is a right upper proximal isometric view of needle drive 130 of device 100. As illustrated, the drive 130 includes the proximal tubular portion 132D that extends toward a proximal end of the device 100 where the tubular portion 132D becomes operably coupled to an actuator 172 (FIG. 53). Tubular portion 132D can be hollow and defines a bore along its length to permit other tools to pass therethrough, as illustrated. Tubular portion 132D widens out into a yoke structure that is defined by a laterally extending portion 132C that in turn terminates in two distally extending arms 132B and 132A. Arm 132B terminates in a pawl 134 that is hook shaped in that it includes a laterally inwardly extending portion 134A that extends laterally inwardly in a distal end region of arm 132B, and a proximally extending portion that can terminate in a proximally directed point or edge 134B that is configured to interfit with one or more indentations of needle 200, discussed in further detail below. Arm 132A terminates in a distal region in pawl 136. As depicted, pawl 136 can be shaped differently from pawl 134, and as illustrated includes an inner laterally facing ramped portion 136A that forms a distal corner with a laterally extending, distally facing, distal wall portion 136B.

In operation, as discussed further below, the drive 130 is configured to advance, and if desired, reciprocate, the drive pawls 134, 136 along a drive pawl pathway wherein at least one of the pawls 134, 136 drives the needle 200 about the needle track 120 along a desired direction of travel that leads with a leading edge 222 of the main needle body 220 or a trailing edge 224 of the main needle body 220. Each respective pawl is configured to engage a surface of the needle while moving the arcuate needle 200 from a starting position to an ending position during a drive stroke. The drive stroke may advance the needle 200 about the needle track 120 while the drive 130 is advancing distally, and/or while the drive 130 is moving proximally.

The drive pawl pathway may be configured to not match, or to deviate from, a curvature of a needle pathway traversed by the arcuate needle, as desired. For example, in operation, depending on the specific configuration of device, 100, the pathway traversed by each pawl 134, 136 can be linear and/or curved. The pathway traversed by one or both pawl scan be linear when not engaging the arcuate needle, and/or when engaging the arcuate needle. If desired, the pathway traversed by one or both pawls can be curved when engaging the arcuate needle and when advancing the arcuate needle. The pawls 134, 136 as illustrated are in a geometric relationship with the arcuate needle 200 that is approximately tangential, wherein the pathway traversed in three-dimensional space by each pawl 134 136 can be linear as the tubular portion 132D of the drive is advanced through the bore 110A of the housing. However, due to tolerancing or desired device performance, the pawls 134, 136 can be pushed laterally outwardly to a slight extent when moving past the needle 200 along a distal or proximal direction by elastic deformation of arms 132A, 132B. Alternatively, the pawl may not be deflected laterally when engaging the needle, such as when pawl 136 pushes a trailing edge 224 of the main needle body portion 220. As described below, during certain portions of a drive stroke the pawl 134 or 136 may need to deflect laterally outwardly to pass or slide over the radially outward edge 221 of the surface of the main needle body 220. As is illustrated, each pawl 134, 136 is laterally offset with respect to a central longitudinal axis of the suturing device wherein the first drive pawl 134 engages the arcuate needle 200 proximate a first lateral side of the arcuate needle track 120, and the second drive pawl 136 engages the arcuate needle 200 proximate a second lateral side of the arcuate needle track 120. When arms 132A, 132B deflect laterally outwardly, they can extend into windows 113A, 113B defined in the housing to prevent the drive 130 from jamming during operation.

In accordance with further implementations, if desired, one or both of the first drive pawl 134 and second drive pawl 136 can traverse a linear pathway when engaging the needle 200. In the illustrated embodiment, the first drive pawl 134 and second drive pawl 136 move in tandem when the drive 130 is advanced proximally or distally since the drive yoke with pawls is monolithically, or integrally, formed. However, in an alternative implementation (not shown), the drive pawls 134, 136 can be mechanically decoupled, wherein each half of the yoke structure is coupled to a different proximal tubular portion that can be moved independently from the other. In addition, in some implementations, the arms 132A and 132B can be of different lengths to alter the point during the drive cycle when pawls 134, 136 engage the needle 200. If desired, in some implementations, the drive pawls 134, 136 can physically contact the arcuate needle 200 simultaneously, or nearly simultaneously, when the arcuate needle is in a predetermined position within the arcuate needle track 120, discussed in further detail below.

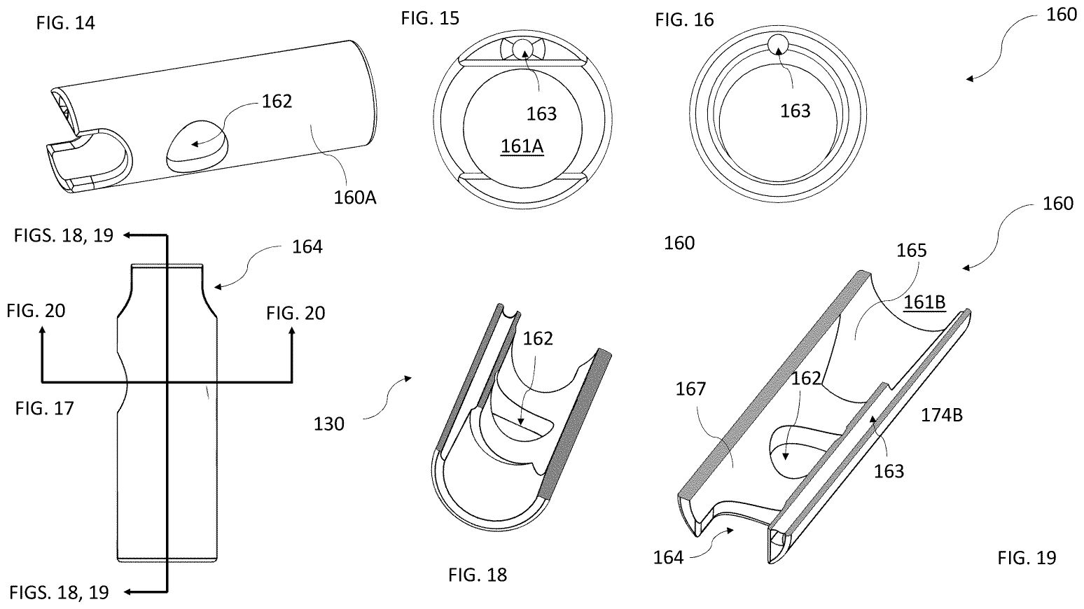

FIGS. 14-22 present various views of a distal end portion 160 of a needle delivery catheter that can removably couple with a proximal section 140 of the catheter. FIG. 15 is a distal end view of the component of FIG. 14. As depicted, distal end portion 160 has an outer shape that is generally cylindrical having a proximal end, a distal end, and an outer generally cylindrical surface.

Distal end portion 160 acts as a fastener (e.g., as a suture anchor or suture cinch) to form a suture loop after a suturing procedure is complete. In use, a distal end of a suture thread (not shown) is attached to a needle tip portion 210 of the needle 200. The suture thread extends from the needle tip portion 210 proximally through passageway 163 of distal end portion 160 toward a proximal end of the device (not shown), wherein the suture thread can be pulled outside of the patient, as desired, or may be maintained internally with respect to the patient. Passageway 163 can be shaped to form a tether crimp or lock that, when tensioned, can capture the suture material. In use, as discussed below, the needle tip 210 can be captured in a lateral opening 162 of distal end portion 160 so that the suture forms a loop extending out of the passageway 162 where the needle resides, around a tissue structure, and proximally through passageway 163. Distal end portion 160 can then be removed from proximal section 140 and left within a patient to complete a suture loop. While a crimp or lock can be incorporated into passageway 163, a suture lock or cinch can alternatively be advanced along the suture material using the suture as a rail. The cinch can then be pushed against distal end portion 160 to tension the suture. A cutting catheter (not shown) can then be advanced along the tether to permit the suture to be cut after the suture is cinched.

With continuing reference to FIGS. 14-25, distal end portion 160 includes a flattened proximal end that defines a generally eccentric conical bore 161B to receive conically shaped distal end surface 145 of proximal portion 140 of the needle delivery catheter. Distal end portion 160 further defines a proximal bore 161A (FIG. 15) that is eccentric with respect to the cylindrical outer surface 160A of distal end portion 160. Bores 161A, 161B are eccentric as indicated to provide sufficient clearance for passageway 163. The distal end of distal end portion 160 terminates in an open transverse bore 164 that is defined by two “U”-shaped openings that intersect a distal end plane of distal end portion 160, thereby forming a “U”-shaped cross section of distal end portion 160 as presented in FIG. 21. The open transverse bore 164 provides a nest or receptacle for a new needle tip 210′ to be delivered to be coupled to the main body portion 220 of the needle 200. With continuing reference to FIG. 19, bore 161A is defined by a generally cylindrical inner wall 167 that extends from a distal end of distal end portion 160 in a proximal direction until it intersects surface 165.

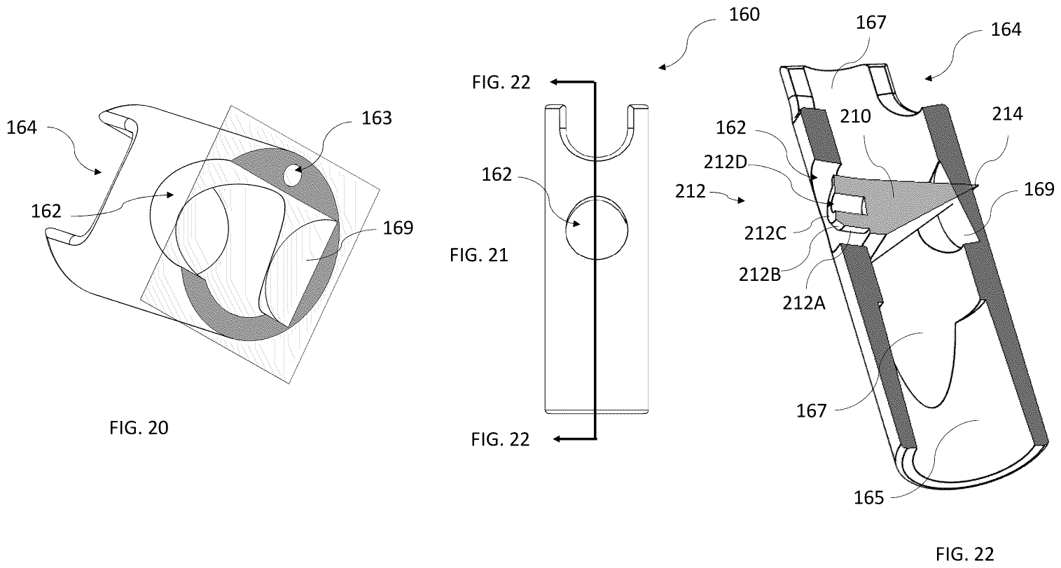

As illustrated in FIG. 20 a transverse bore 162 is defined transversely across distal end portion 160. Transverse bore 162 provides an opening for a new needle tip 210′ to be delivered to be coupled to the main body portion 220 of the needle 200. Bore 162 intersects bore 161A at approximately a right angle. Bore 162 is defined by an opening at one lateral side of component that extends laterally across distal end portion 160 to a side wall 169. The material of the side wall 169 can be made of a material that is sufficiently soft to permit penetration of the distal end 214 of the needle tip (FIG. 22) to engage the distal end 214 of the needle tip portion 210 within the side wall of distal end portion 160. In this manner, the main body portion 220 of the needle 200 can be disengaged and retracted from the needle tip, discussed in further detail below.

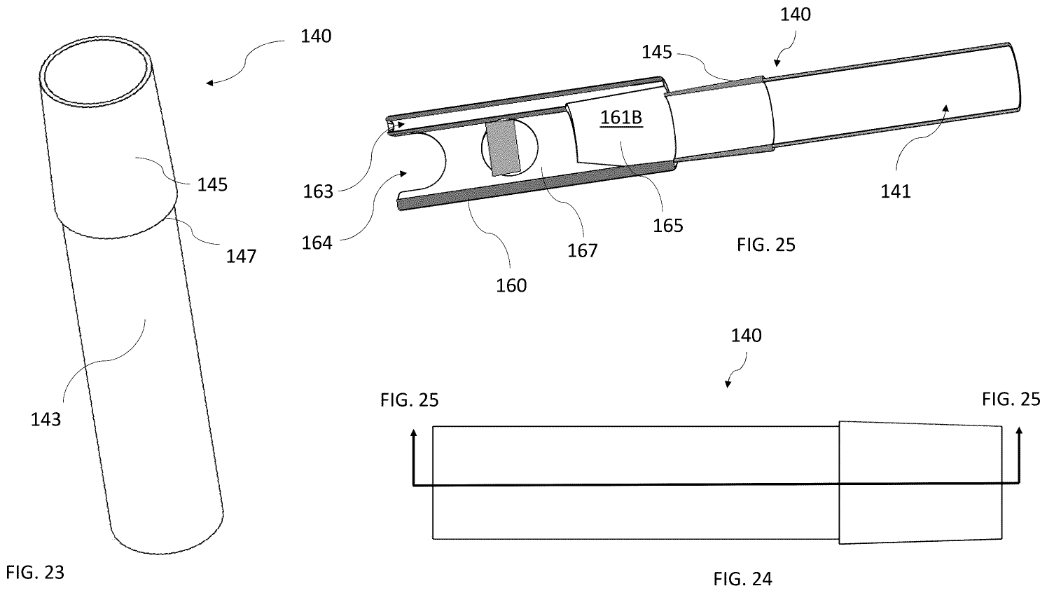

As illustrated in FIG. 23, the proximal section 140 of the needle delivery catheter is defined by an elongate proximal tubular portion 143 that defines a generally cylindrical bore 141 along its length from the proximal end of proximal section to the distal end of proximal section 140. Proximal section 140 terminates in a conical tip that has the same inner diameter as proximal portion 143 of proximal section 140 but that has an enlarged proximal ridge that steps down in radius at the juncture of components 145 and 143. As illustrated in FIG. 25, passageway 163 that extends along the length of distal end portion 160 begins at a distal end of distal end portion 160 and intersects bore 161B inside of bore 161B.

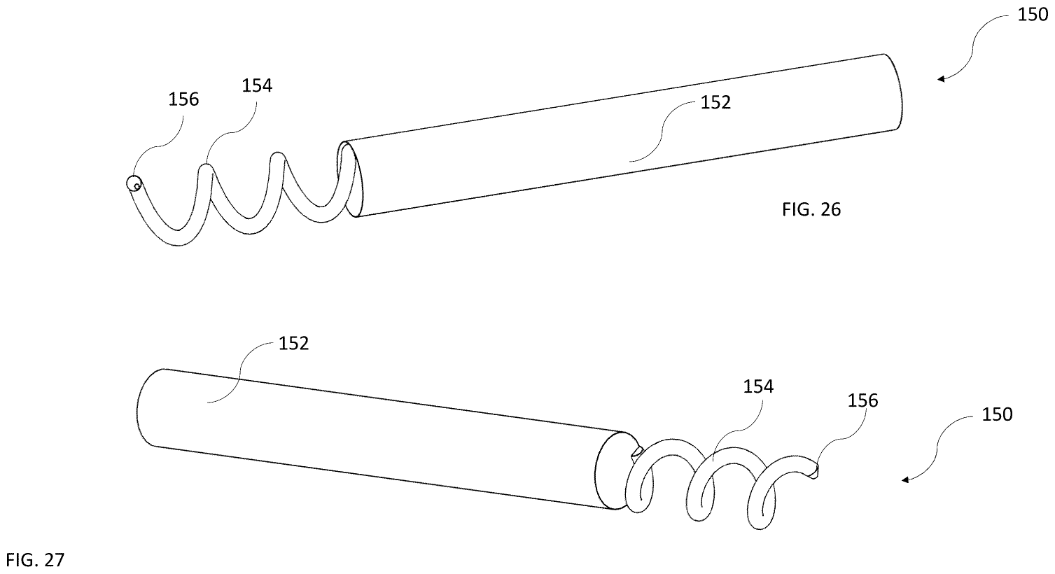

FIGS. 26-27 illustrate isometric views of a distal end portion of a tissue capture catheter 150 that is slidably received within bore or passageway 141 of proximal section 140. The illustrated catheter 150 includes a cylindrical proximal portion 152 that transitions into a helical portion 154 (e.g., a tissue helix) that terminates in a tissue piercing distal tip 156. Tissue capture catheter 150 can be extended out through bore 141 into the tissue gap 112 of device 100, and may be rotated in a counterclockwise direction (as viewed along a distal direction) while pressed against tissue to advance into the tissue. The tissue can then be pulled toward the tissue gap by retracting catheter 150 along a proximal direction. To avoid colliding with the needle 200 while the needle is present in the needle track 120, the tissue capture catheter 150 can be a shape memory alloy, or a polymeric material imparted with a shape memory to cause the catheter 150 to bend off of its axis as it emerges out of the distal end of the passageway 141 into the tissue gap. For example, the distal region of proximal portion 152 may be a polymeric material that is heated and bent to impart a bend thereto prior to introduction into suturing device 100. This can result in the distal end 156 of the tissue capture catheter to pass over or under the needle track as the tissue capture catheter 150 passes past the distal end of the device 100, which can cause the catheter to proceed over or under the needle 200. By way of further example, a second passageway can be defined in housing (not shown) that is parallel to and underneath passageway 141 to receive the tissue capture catheter 150. In a further implementation, the tissue capture catheter 150 can be provided within a working channel of an endoscope (e.g., 300, FIG. 53) that is coupled to suturing device 100 or used alongside suturing device 100.

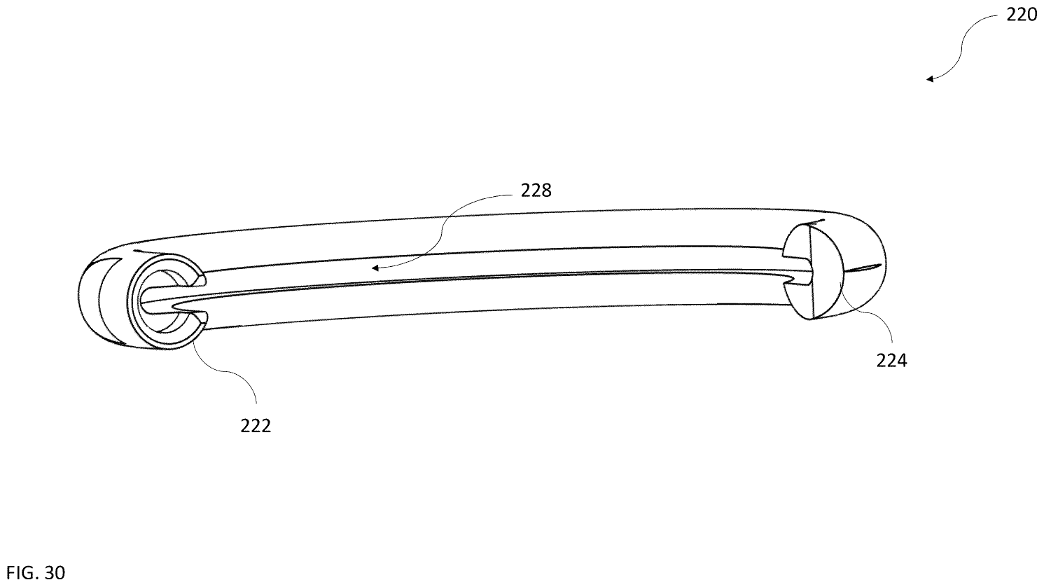

FIGS. 28-33 illustrate aspects of an arcuate needle 200 in accordance with the present disclosure that can be used within suturing device 100.

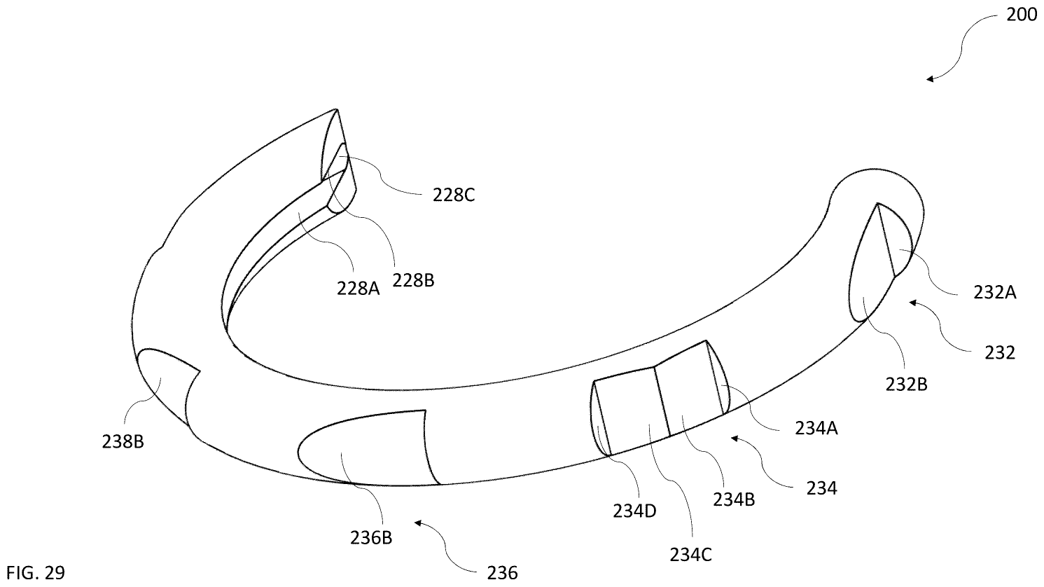

As illustrated, needle includes a main body portion 220. The main body portion 220 is defined by a leading end 222, a trailing end 224, and an arcuately shaped body. As illustrated, the arcuately shaped body defines a bore 226 along its length from the leading end 222 of the main body portion 220 to the trailing end 224 of the main body portion 220. The bore 226 extends radially inwardly through an inner radial wall of the arcuately shaped body to form an elongate slit 228, such that the main body portion 220 has a “C” shaped cross section along its length. With reference to FIG. 29, slit 228 and the bore 226 are defined by a lower annularly shaped planar surface 228A and an upper annularly shaped planar surface 228B joined by a concave toroidal surface 228C.

Main body portion includes a radially outer surface 221, a radially inner surface 223, a radially upper, or “top” surface 225, and a radially lower surface 227 (FIG. 33). As illustrated in FIG. 33, the trailing edge 224 of the main body portion 220 can be located adjacent a chamfered surface 229 to reduce interference of the trailing end 224 of the needle with instrumentalities that are introduced along the central passageway of the device, such as tissue capture catheter 150.

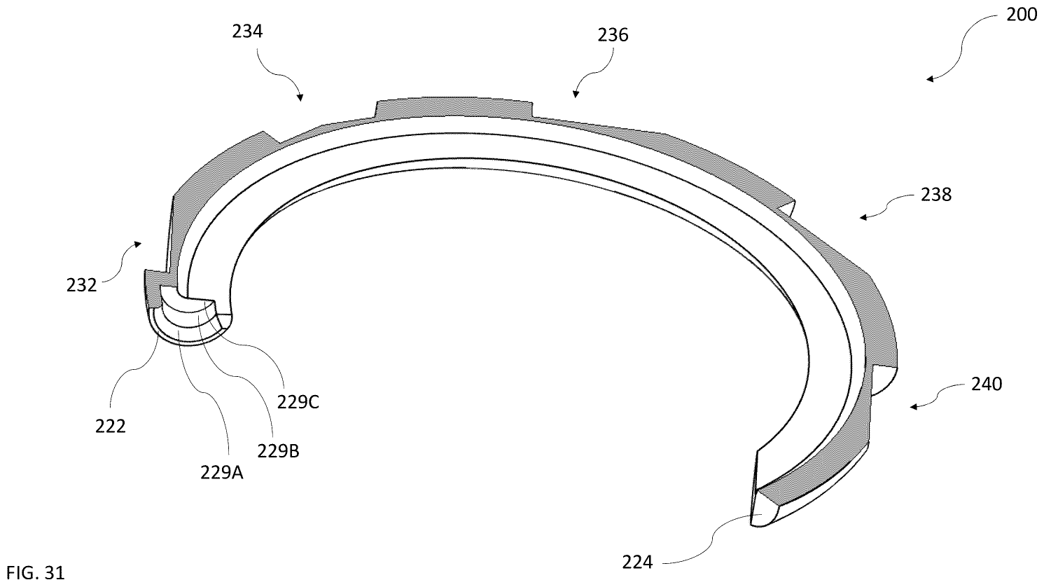

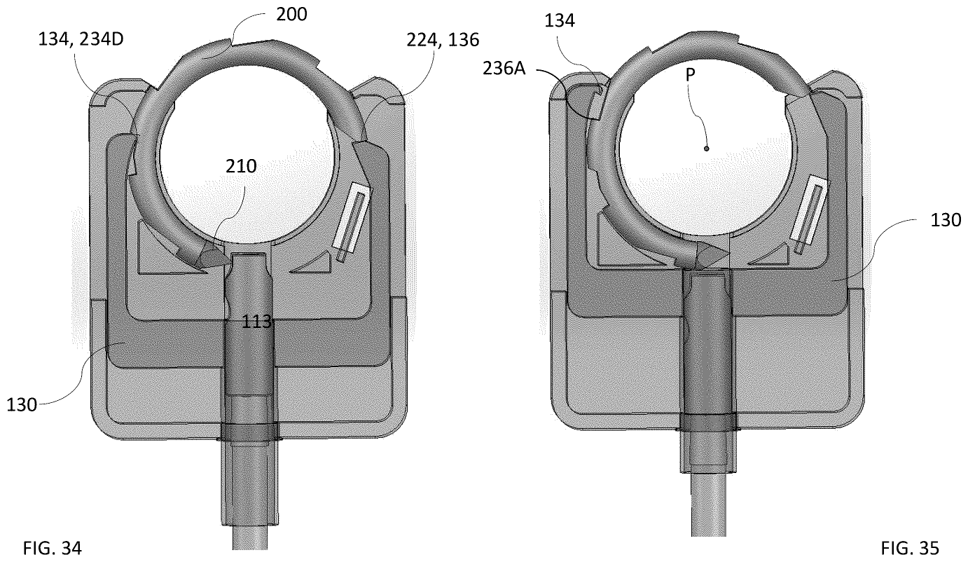

As illustrated in FIGS. 22 and 34, for example, the arcuate needle 200 can further include a needle tip portion 210 that is removably coupled to the leading end 222 of the main body portion 220. The needle tip portion 210 includes a pointed distal end 214 and a proximal end 212 terminating in a proximal coupling to couple to the main body portion 220. Specifically, as is visible in FIG. 22, for example, the proximal coupling of the needle tip portion 210 can include a boss configured to be received by a cavity defined in the leading end of the main body portion of the needle. As illustrated in FIG. 22, the boss is annularly shaped and defines a central bore 212D to receive a distal end of length of suturing material (not shown) that extends proximally from the boss located at the proximal end 212 of the needle tip portion 210 into the bore 226 of the main body portion 220. With reference to FIG. 31, the main body portion 220 of the needle 200 defines a generally cylindrical cavity at leading end 222 of the main body portion 220 that is formed from annular surface 229A that receives outer cylindrical surface 212A of the proximal end 212 of the needle tip portion 210. Proximal end 212 of the needle tip portion 210 is further defined by an angled annular surface 212B that is received by a corresponding surface 229B of the main body portion 220. Surface 229C of the main body portion 220 in turn receives flattened proximally facing surface 212C of the needle tip portion 210.

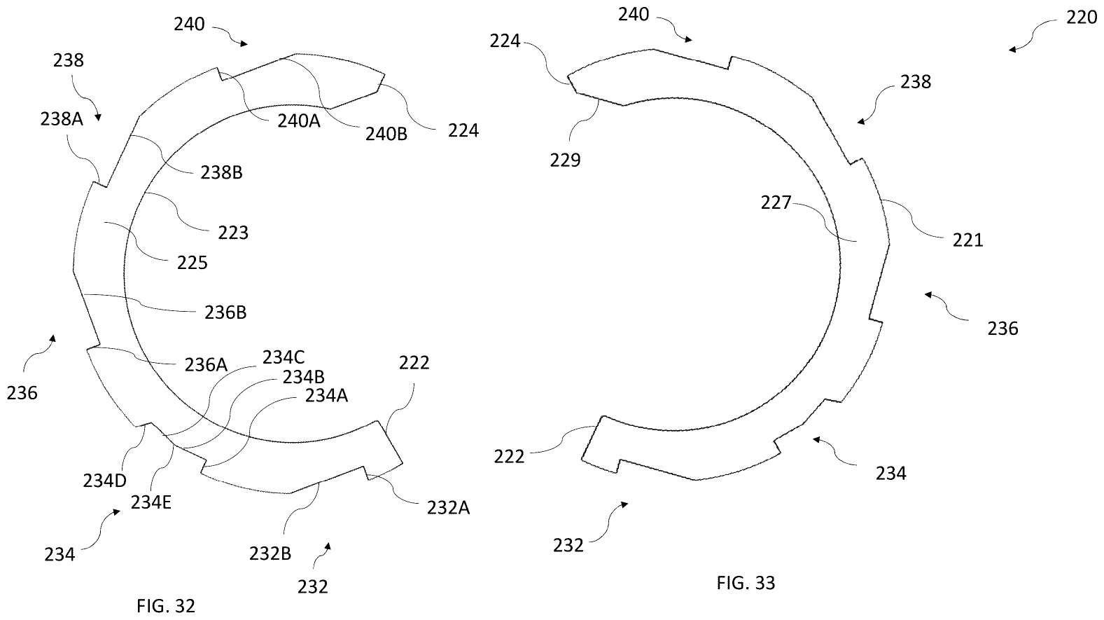

As illustrated in FIG. 32, the outer radial surface 221 of the main body portion 220 of the arcuate needle 200 can define one or more indentations or notches 232, 234, 236, 238, 240 therein. Each indentation, as illustrated, is defined by at least one facet (e.g., 232A, 234A, 236A, 238A, 240A) laying in a plane that is transverse to a central arcuate axis of the main body portion 220 of the needle 200. As is further illustrated, each of indentations, or notches, 232, 236, 238, 240 is formed by two intersecting facets (e.g., 232A, 232B; 236A, 236B; 238A, 238B; 240A, 240B). At least one of said intersecting facets (e.g., 232B; 236B; 238B; 240B) can lay in a plane that is oblique to a central arcuate axis of the main body portion of the needle. If desired, at least one of the indentations (e.g., 234) can include a lower surface formed by facets 234B, 234C that cooperate to define a peak 234E along a bottom of the indentation 234. Indentation 234 is symmetrical about peak 234E and is defined by facets 234A, 234B, 234C and 234D. Facets 234A, 234D form two opposing sidewalls of indentation 234. In some implementations, at least two of the indentations (e.g., 232, 240) can include portions that are located antipodally, diametrically across the main body portion 220 of the needle 200 from one another.

FIGS. 34-44 illustrate steps of operation of the device of FIG. 1 to advance an arcuate needle fully about a needle track of the device.

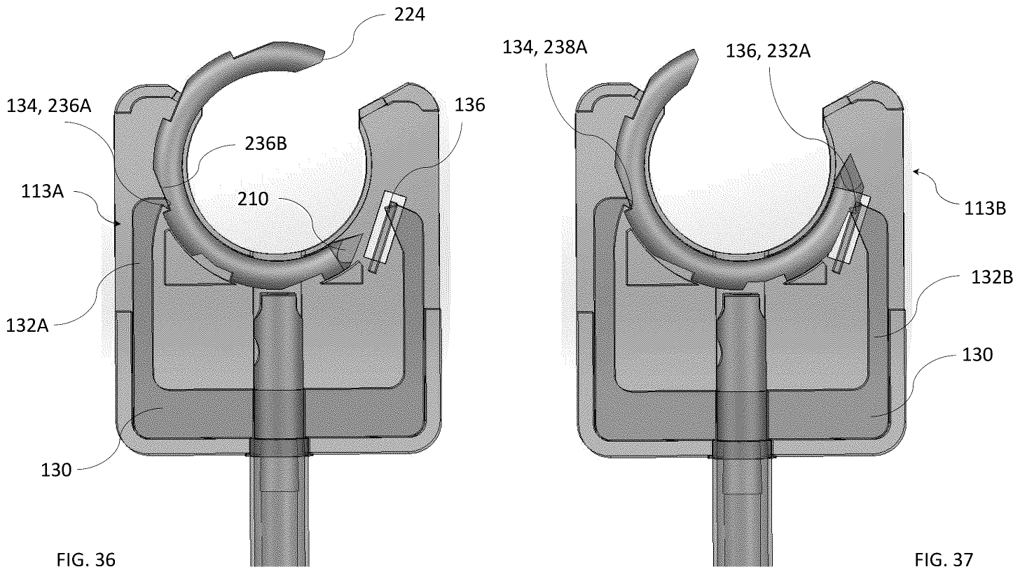

For purposes of illustration, and not limitation, drive pawl 136 can be configured to advance the arcuate needle 200 along a first rotational direction (counterclockwise in the referenced figures) along the arcuate needle track 120 when the drive pawl 136 is advanced along a distal direction that is generally linear, and parallel to a central longitudinal axis of the device 100. As illustrated in FIGS. 34-35, when beginning a rotation from the position indicated in FIG. 34, drive 130 can be advanced distally wherein the distal wall portion 136B of pawl 136 contacts the trailing end 224 of the needle 200, resulting in the needle 200 being advanced in a counterclockwise direction. Proceeding from FIGS. 35 to 36, drive 130 is retracted proximally such that the pointed proximally facing edge 134B of pawl 134 engages surface 236A of needle 200. This proximal movement of the drive 130 and pawl 134 therefore continues to advance needle along a counterclockwise direction as illustrated in FIG. 36.

In the position of FIG. 36, needle drive 130 has reached its proximal-most position. From this position, the drive 130 can be advanced distally again in a further drive stroke permitting pawl 134 to pass distally over the ramped surface 236B of indentation 236, causing arm 132A of drive 130 to splay laterally outwardly (not shown) into window 113A defined in the side of housing 110. This permits pawl 134 to engage surface 238A of indentation 238 as the drive 130 is again withdrawn proximally as illustrated in FIG. 37. As the drive 130 and pawl 134 are withdrawn proximally, the needle 200 continues its counterclockwise progression, and the sharpened leading tip of the needle 200 passes by pawl 136, which can correspondingly cause arm 132B to splay laterally outwardly (not shown) into window 113B of housing 110. In this respect, during this step, pawl 134 is withdrawn along a linear path during the drive stroke without noticeable outward splaying of arm 132A.

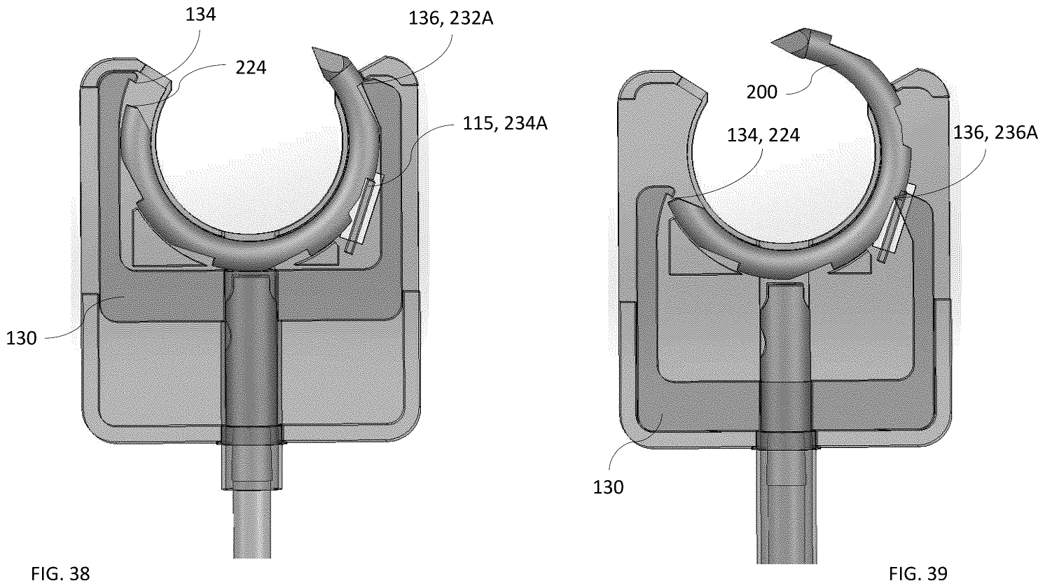

Proceeding from FIG. 37 to FIG. 38, pawl 136 engages facet 232A of indentation 232 and continues to advance the needle along a counterclockwise direction as the drive 130 is advanced distally within the housing 110. In the position as illustrated in FIG. 38, the needle 200 is largely contained within the needle track 120 except for the needle tip portion 210 which protrudes from the second end 124 of the needle track 120.

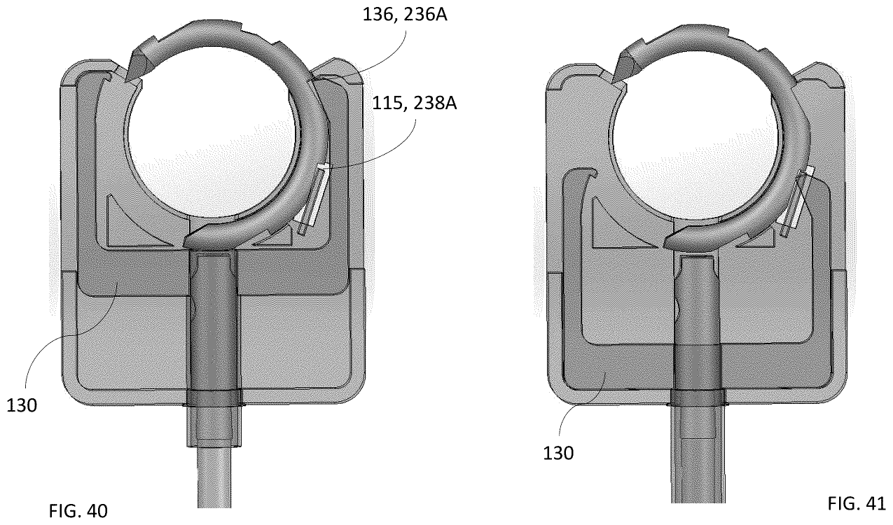

As further illustrated in FIG. 38, braking pawl 115 engages surface 234A of indentation 234, which prevents the needle 200 from moving in a clockwise direction as pawl 136 is withdrawn proximally, ending in the position presented in FIG. 39. To achieve movement from FIG. 38 to FIG. 39, pawl 134 engages, grips, and pulls trailing edge 224 of main body portion 220 of needle 200 proximally, and thus continues the counterclockwise motion of needle 200. As will be appreciated from FIG. 39, as drive 130 bottoms out in the housing 110, pawl 136 slides over the outer radial surface 221 of the needle 200 and snaps into indentation 236, and engages surface 236A. Thus, as the drive 130 moves distally again, the needle 200 is again advanced in a counterclockwise direction from the position illustrated in FIG. 39 to the position illustrated in FIG. 40. When the needle 200 has reached the location illustrated in FIG. 40, braking pawl 115 slides over the outer radial surface 221 of the needle 200 and snaps into indentation 238. The distal end of the braking pawl 115 is configured to contact surface 238A to prevent the needle from moving in a clockwise (backward) direction along the needle track 120 as the drive 130 is cycled proximally as shown in FIG. 41.

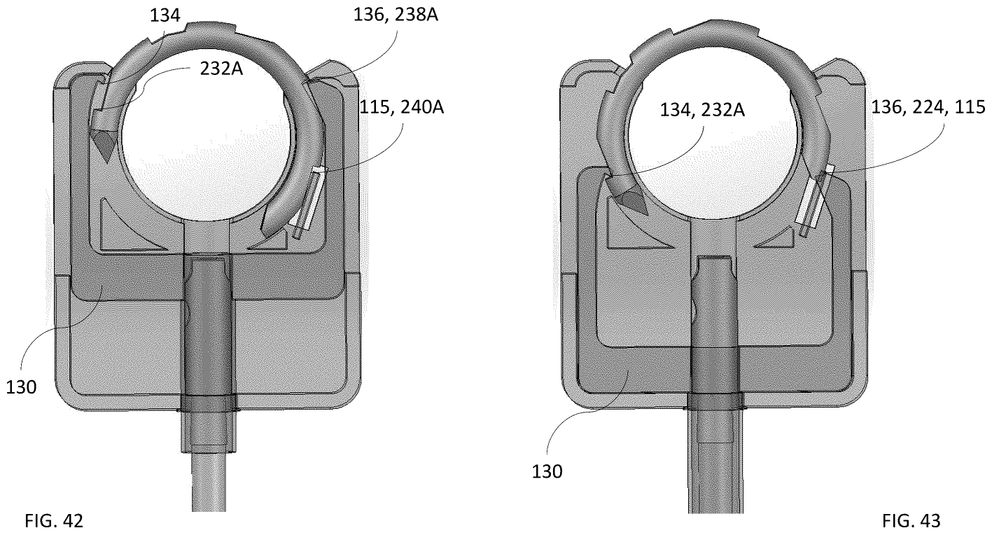

In addition to engaging the braking pawl 115, in the orientation of FIG. 41, as the pawl 136 is again advanced distally, its lateral inward bias forces the lateral inward corner, or point of pawl 136 in between surface 238A of indentation 238, and the free end of the braking pawl, permitting pawl to engage surface 238A, and push surface 238A distally to further advance the needle from the position shown in FIG. 41 to the position illustrated in FIG. 42.

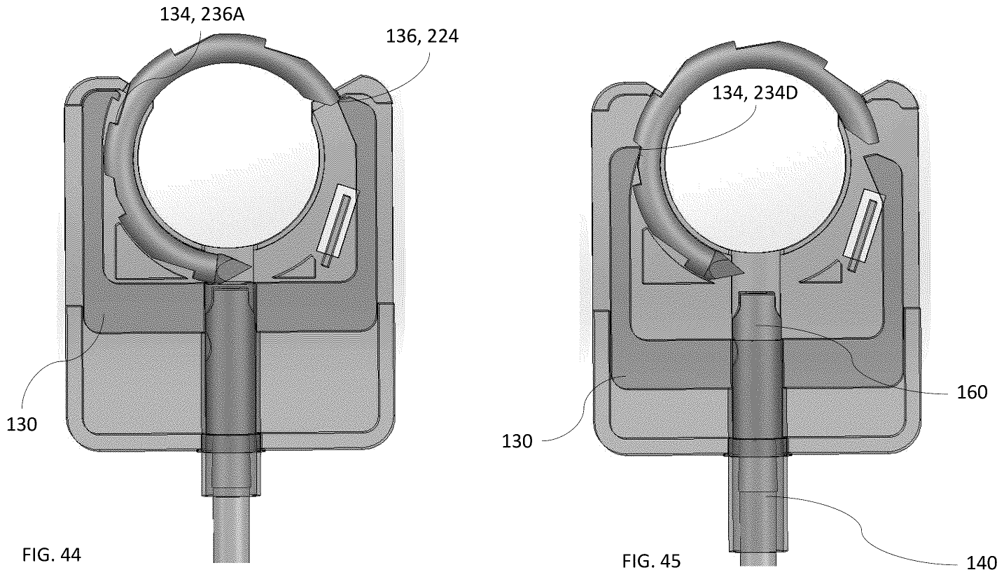

With continuing reference to FIG. 42, the drive 130 is in its distal-most position within the housing 110, and braking pawl 115 has engaged indentation 240, such that clockwise (backward) motion of the needle 200 is prevented by mechanical interference between surface 240A and the distal free end of the braking pawl 115. As further illustrated in FIG. 42, in this position, pawl 134 is located distally of surface 232A of indentation 232. This permits pawl 134 to again pull surface 232A along a proximal direction as the drive 130 is again withdrawn proximally to the position shown in FIG. 43. It will be further noted that, when reaching the position of FIG. 43, the needle 200 is prevented from moving in a clockwise direction by virtue of the free end of the braking pawl 115 snapping in place behind the trailing end 224 of the needle 200. This permits the inward pointed edge of the pawl 136 to push the trailing end 224 of the needle 200 distally, separating the needle 200 from contact with the free end of the braking pawl 115. The pawl 136 therefore pushes on the trailing end 224 of the needle 200, continuing to advance the needle in a counterclockwise direction until the needle 200 has reached the position indicated in FIG. 44. In this position, the drive 130 may be withdrawn proximally, and needle movement can be continued.

In the implementation illustrated in FIGS. 34-52, the drive 130 is configured to advance the drive pawls 134, 136 from a proximal starting position, represented, for example, in FIG. 36, along a linear path prior to engagement with the needle 200. As further illustrated, the starting position of the drive pawls 134, 136 is located proximally with respect to a center point P (FIG. 35) of the arcuate needle track 120, which is located at the geometric center of the path traced out by the needle 120 as it traverses the needle track 120. As further illustrated, the drive 130 is configured to advance the drive pawl along a linear path after disengagement with the needle 200 until the drive pawls 134, 136 reaches the ending position, illustrated or example, in FIG. 38. As illustrated, the ending position of the drive pawl is located distally with respect to center point P of the arcuate needle track.

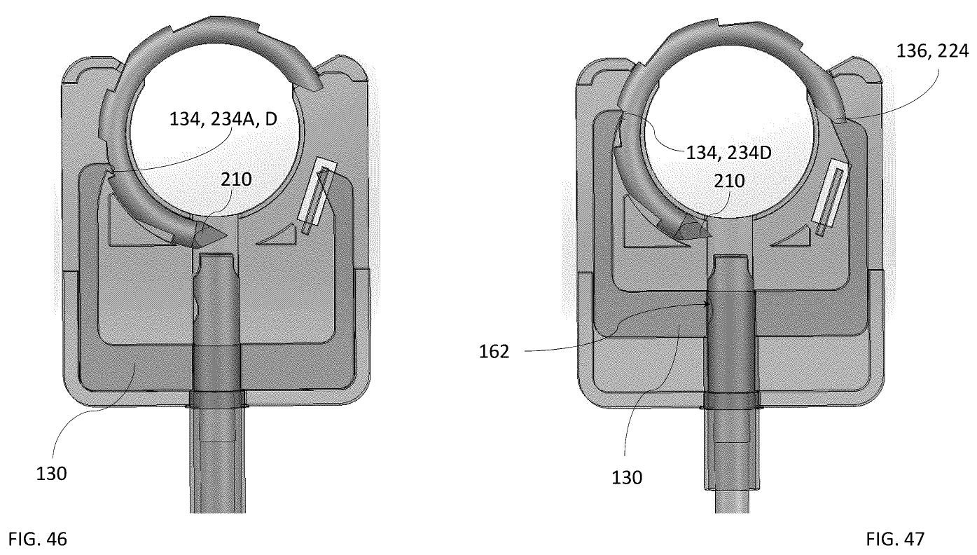

FIGS. 45-50 illustrate aspects of removing a needle tip from a main body portion 220 of a needle in accordance with the present disclosure. FIG. 45 illustrates how pawl 134 can engage surface 234D of notch 234 to reverse the direction of travel of the needle 200 to a clockwise direction as the pawl 134 is advanced in a distal direction. This movement is facilitated by facets 234C, 234D formed into indentation 234. Also illustrated in FIG. 45 is relative sliding advancement of distal end portion 160 which is advanced via coupling with proximal section 140. FIGS. 46 and 47 further illustrate how the interaction of pawl 134 with indentation 234 can permit the needle 200 to be moved forward and backward with drive 130 to direct the removable needle tip 210 into transverse bore 162 of distal end portion 160.

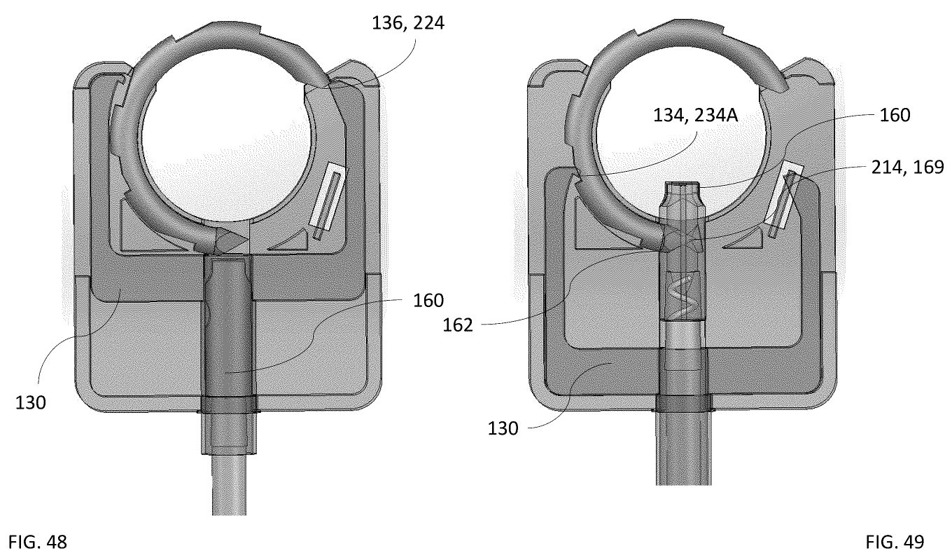

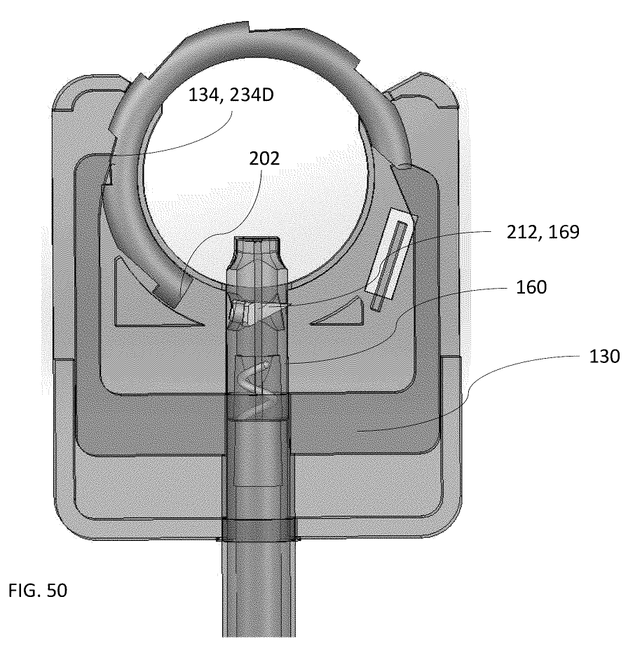

FIGS. 48 and 49 illustrate how distal advancement of pawl 136 urges the trailing end 224 of the needle 200 to drive the needle tip into bore 162 of distal end portion 160 when bore 162 is aligned with the needle track 120. Once the needle tip 210 is pushed into the bore 162, the tip 210 of the needle 200 anchors itself into the material of the opposing side wall 169 (FIG. 22). With the needle tip being stuck in this manner, and/or by way of an interference fit with the bore 162, clockwise movement of the main body portion 220 of the needle 200 can detach the main body portion 220 from the boss formed on the proximal end of the needle tip 210 as indicated in FIG. 50. In this position, the suture material (not shown) has been routed through tissue of a patient to form a loop that extends from the needle tip, through the tissue, and back through passage 163 of distal end portion 160. A cinch or lock can be defined by passage 163, or a separate lock or cinch can be advanced along a proximal end of the suture until the cinch or lock mechanically contacts a proximal end of distal end portion 160. Distal end portion 160 can then be separated from proximal section 140, and can be left in the patient, forming a complete suture loop.

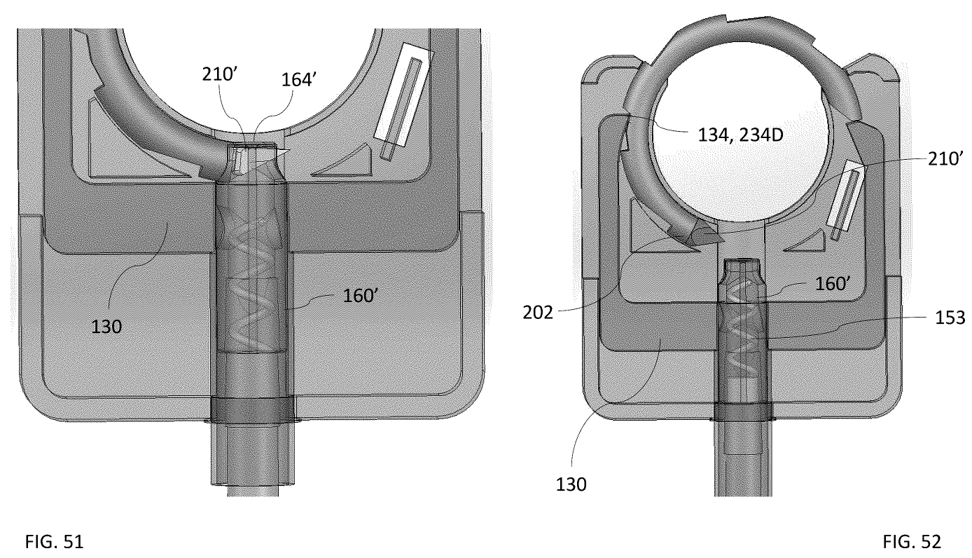

FIGS. 51-52 illustrate how a new distal end portion 160′ bearing a new needle tip 210′ in distal cradle or bore 164′ can be attached to proximal section 140 and re-inserted into the longitudinal bore of the device 100. The bore 164 can be aligned with the needle track 120, and the needle drive 130 can be pulled proximally, forcing the leading end 222 of the main body portion of the needle 200 into an interlocking relationship with the new tip 210′, which includes a new portion of suture thread (not shown) extending therefrom into the bore 226 of the main body portion of the needle, and proximally through the passageway 163′ (not shown) in distal end portion 160.

Also illustrated in FIG. 52 is relative placement of tissue capture catheter 150. Tissue capture catheter 150 can be advanced through the bore defined in proximal section 140 and distal end portion 160 distally past the needle track to capture tissue to be sutured as described above.

In further accordance with the disclosure, the suturing device can further include a coupling to receive and couple to a distal end of an endoscope or catheter.

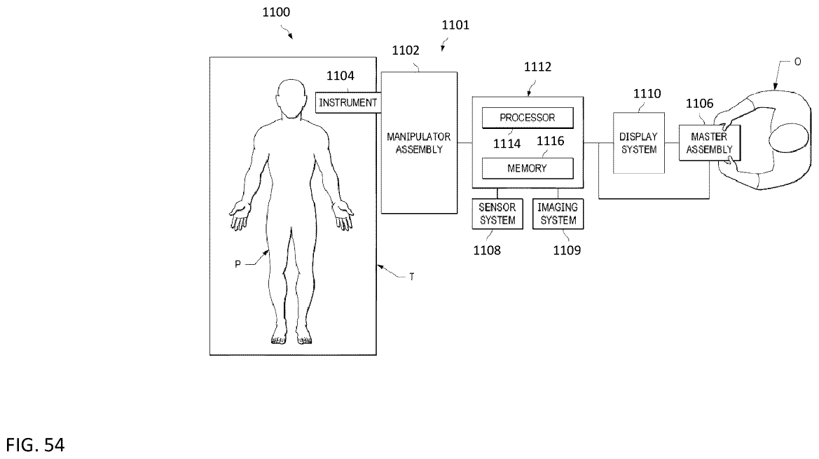

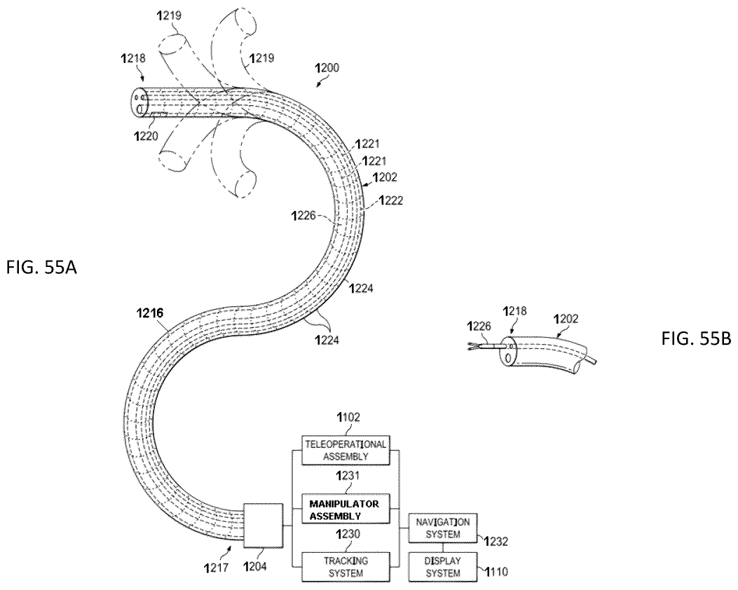

For purposes of illustration, and not limitation, as depicted in FIG. 53, suturing device 100 can be coupled to a proximal device portion that terminates in an actuator system 170. The suturing device 100 can be further coupled to an endoscope 300, as desired. The endoscope 300 can provide one or more working channels (not shown) and may include an imaging system (e.g., including a camera) to provide direct visualization of the working area, as described in further detail with respect to FIGS. 54-55B. The endoscope 300 may provide a channel to receive tissue capture catheter 150, and if desired, the endoscope 300 may be oriented obliquely with respect to the arcuate needle to provide enhanced visualization of a suturing procedure. This facilitates viewing the passage of the needle across the gap 112 using a camera(s) disposed in one (or more) of a plurality of passageways defined along the length of the endoscope 300. Endoscope 300 may be a conventional endoscope, or an endoscope that is specially configured for use with suturing device 100.

With continuing reference to FIG. 53, as mentioned above, the system can include an actuator system 170, wherein drive 130 includes a proximal tubular portion 132D that extends toward a proximal end of the device 100 where the tubular portion 132D becomes operably coupled to an actuator 172. Proximal section 140 can similarly extend proximally to an actuator 174, and tissue capture catheter 150 can similarly extend proximally to an actuator 176.

The disclosed suturing devices can be used to perform various suturing procedures, such as laparoscopic, robotically-assisted minimally invasive procedures, and other procedures. In some implementations, it can be advantageous to provide an additional tool such as tissue capture device 150 in suturing device 100 to help capture and pull tissue into gap 112 to suture. This can be particularly useful when the tissue being sutured is not compliant, or when it is desired to grasp a planar surface that would not naturally fall into the tissue gap 112, and it is necessary to form a fold, or plication of such a tissue structure to be pulled into gap 112. Implementations of devices in accordance with the present disclosure can be used to perform an Endoscopic Sleeve Gastroplasty (“ESG”) procedure, wherein a device 100 can be deployed into a patient's stomach, and elongate tool 150 can be used to grab portions of the stomach lining to form a plication, and the plication can then be held in place by passing needle 200 through the plication. This procedure can be repeated until all desired plications are formed. Tension can then be applied to the suture passing through the plications to reduce the volume of the stomach.

Aspects of the suturing device 100 described herein can be part of a computer-assisted teleoperational manipulator system, sometimes referred to as a robotically-assisted manipulator system or a robotic system. The manipulator system can include one or more manipulators that can be operated with the assistance of an electronic controller (e.g., computer) to move and control functions of one or more instruments when coupled to the manipulators.

FIG. 54 illustrates an embodiment of a robotically-assisted manipulator system for use with the suturing devices described herein. The manipulator system can be used, for example, in surgical, diagnostic, therapeutic, biopsy, or non-medical procedures, and is generally indicated by the reference numeral 1100. As shown in FIG. 54, a robotically-assisted manipulator system 1100 can include one or more manipulator assemblies 1102 for operating one or more medical instrument systems 1104 in performing various procedures on a patient P positioned on a table T in a medical environment 1101. For example, the manipulator assembly 1102 can drive catheter or end effector motion, can apply treatment to target tissue, and/or can manipulate control members. The manipulator assembly 1102 can be teleoperated, non-teleoperated, or a hybrid teleoperated and non-teleoperated assembly with select degrees of freedom of motion that can be motorized and/or teleoperated and select degrees of freedom of motion that can be non-motorized and/or non-teleoperated. An operator input system 1106, which can be inside or outside of the medical environment 1101, generally includes one or more control devices for controlling manipulator assembly 1102. Manipulator assembly 1102 supports medical instrument system 1104 and can optionally include a plurality of actuators or motors that drive inputs on medical instrument system 1104 in response to commands from a control system 1112. The actuators can optionally include drive systems that when coupled to medical instrument system 1104 can advance medical instrument system 1104 into a naturally or surgically created anatomic orifice. Other drive systems can move the distal end of medical instrument in multiple degrees of freedom, which can include three degrees of linear motion (e.g., linear motion along the X, Y, Z Cartesian axes) and in three degrees of rotational motion (e.g., rotation about the X, Y, Z Cartesian axes). The manipulator assembly 1102 can support various other systems for irrigation, treatment, or other purposes. Such systems can include fluid systems (including, for example, reservoirs, heating/cooling elements, pumps, and valves), generators, lasers, interrogators, and ablation components. The suturing devices (e.g., 100) described herein are an example of the medical instrument system 1104, and the suturing devices 100 may have a substantially rigid shaft or may have a shaft that is a substantially flexible member.

Robotically-assisted manipulator system 1100 also includes a display system 1110 for displaying an image or representation of the surgical site and medical instrument system 1104 generated by an imaging system 1109 which can include an imaging system, such as an endoscopic imaging system. Display system 1110 and operator input system 1106 can be oriented so an operator O can control medical instrument system 1104 and operator input system 1106 with the perception of telepresence. A graphical user interface can be displayable on the display system 1110 and/or a display system of an independent planning workstation.

In some examples, the endoscopic imaging system components of the imaging system 1109 can be integrally or removably coupled to medical instrument system 1104. However, in some examples, a separate imaging device, such as an endoscope, attached to a separate manipulator assembly can be used with medical instrument system 1104 to image the surgical site. The endoscopic imaging system 1109 can be implemented as hardware, firmware, software, or a combination thereof which interact with or are otherwise executed by one or more computer processors, which can include the processors of the control system 1112. It will be appreciated that either an integrated endoscopic device or a separate endoscope can serve as an endoscope that supports the suturing device (e.g., 100) set forth herein.

Robotically-assisted manipulator system 1100 can also include a sensor system 1108. The sensor system 1108 can include a position/location sensor system (e.g., an actuator encoder or an electromagnetic (EM) sensor system) and/or a shape sensor system (e.g., an optical fiber shape sensor) for determining the position, orientation, speed, velocity, pose, and/or shape of the medical instrument system 1104. The sensor system 1108 can also include temperature, pressure, force, or contact sensors or the like.

Robotically-assisted manipulator system 1100 can also include a control system 1112. Control system 1112 includes at least one memory 1116 and at least one computer processor 1114 for effecting control between medical instrument system 1104, operator input system 1106, sensor system 1108, and display system 1110. Control system 1112 also includes programmed instructions (e.g., a non-transitory machine-readable medium storing the instructions) to implement a procedure using the robotically-assisted manipulator system including for navigation, steering, imaging, engagement feature deployment or retraction, applying treatment to target tissue (e.g., via the application of energy), or the like.

Control system 1112 can optionally further include a virtual visualization system to provide navigation assistance to operator O when controlling medical instrument system 1104 during an image-guided surgical procedure. Virtual navigation using the virtual visualization system can be based upon reference to an acquired pre-operative or intra-operative dataset of anatomic passageways. The virtual visualization system processes images of the surgical site imaged using imaging technology such as computerized tomography (CT), magnetic resonance imaging (MRI), fluoroscopy, thermography, ultrasound, optical coherence tomography (OCT), thermal imaging, impedance imaging, laser imaging, nanotube X-ray imaging, and/or the like. The control system 1112 can use a pre-operative image to locate the target tissue (using vision imaging techniques and/or by receiving user input) and create a pre-operative plan, including an optimal first location for performing treatment. The pre-operative plan can include, for example, a planned size to expand an expandable device, a treatment duration, a treatment temperature, and/or multiple deployment locations.

FIG. 55A shows a medical instrument system 1200 according to some embodiments. In some embodiments, medical instrument system 1200 can be used in an image-guided medical procedure. In some examples, medical instrument system 1200 can be used for non-teleoperational exploratory procedures or in procedures involving traditional manually operated medical instruments, such as endoscopy. In some embodiments, medical instrument system 1200 is interchangeable with, or a variation of, medical instrument system 1104 of FIG. 54.

Medical instrument system 1200 includes elongate flexible device 1202, such as a flexible catheter or endoscope (e.g., gastroscope, bronchoscope), coupled to a drive unit 1204. Elongate flexible device 1202 includes a flexible body 1216 having proximal end 1217 and distal end, or tip portion, 1218. In some embodiments, flexible body 1216 has an approximately 14-20 mm outer diameter. Other flexible body outer diameters can be larger or smaller. Flexible body 1216 can have an appropriate length to reach certain portions of the anatomy, such as the lungs, sinuses, throat, or the upper or lower gastrointestinal region, when flexible body 216 is inserted into a patient's oral or nasal cavity. The suturing device (e.g., 100) can be coupled to elongate flexible device 1202 as desired in lieu of endoscope 300.

Medical instrument system 1200 optionally includes a tracking system 1230 for determining the position, orientation, speed, velocity, pose, and/or shape of distal end 1218 and/or of one or more segments 1224 along flexible body 1216 using one or more sensors and/or imaging devices. The entire length of flexible body 1216, between distal end 1218 and proximal end 1217, can be effectively divided into segments 1224. Tracking system 1230 can optionally be implemented as hardware, firmware, software or a combination thereof which interact with or are otherwise executed by one or more computer processors, which can include the processors of control system 1112 in FIG. 54.

Tracking system 1230 can optionally track distal end 1218 and/or one or more of the segments 1224 using a shape sensor 1222. In some embodiments, tracking system 1230 can optionally and/or additionally track distal end 1218 using a position sensor system 1220, such as an electromagnetic (EM) sensor system. In some examples, position sensor system 1220 can be configured and positioned to measure six degrees of freedom, e.g., three position coordinates X, Y, Z and three orientation angles indicating pitch, yaw, and roll of a base point or five degrees of freedom, e.g., three position coordinates X, Y, Z and two orientation angles indicating pitch and yaw of a base point.

Flexible body 1216 includes one or more channels 1221 sized and shaped to receive one or more medical instruments 1226. In some embodiments, flexible body 1216 includes two channels 1221 for separate instruments 1226, however, a different number of channels 1221 can be provided. FIG. 55B is a simplified diagram of flexible body 1216 with medical instrument 1226 extended according to some embodiments. In some embodiments, medical instrument 1226 can be used for procedures and aspects of procedures, such as suturing, surgery, biopsy, ablation, mapping, imaging, illumination, irrigation, or suction. Medical instrument 1226 can be deployed through channel 1221 of flexible body 1216 and used at a target location within the anatomy. Medical instrument 1226 can include, for example, suturing devices, image capture devices, biopsy instruments, ablation instruments, catheters, laser ablation fibers, and/or other surgical, diagnostic, or therapeutic tools. Medical tools can include end effectors having a single working member such as a scalpel, a blunt blade, a lens, an optical fiber, an electrode, and/or the like. Other end effectors can include, for example, suturing devices, forceps, graspers, balloons, needles, scissors, clip appliers, and/or the like. Other end effectors can further include electrically activated end effectors such as electrosurgical electrodes, transducers, sensors, imaging devices and/or the like. Medical instrument 1226 can be advanced from the opening of channel 1221 to perform the procedure and then retracted back into the channel when the procedure is complete. Medical instrument 1226 can be removed from proximal end 1217 of flexible body 1216 or from another optional instrument port (not shown) along flexible body 1216. The medical instrument 1226 can be used with an image capture device (e.g., an endoscopic camera) also within the elongate flexible device 1202. Alternatively, the medical instrument 1226 can itself be the image capture device. In some embodiments, the suturing device 100 described herein is an example of the medical instrument 1226. For example, the suturing device 100 may have a flexible shaft configured to be extended through the channel 1221 of the flexible body 1216 or extended outside and along a length of the flexible body 1216. In some embodiments, the suturing device 100 can be formed as an attachment configured to be attached to a distal end portion of the flexible body 1216, with the housing 110 extending distally from the flexible body 1216. In such embodiments, the drive 130 may be extended through a channel 1221, while in other embodiments, the drive 130 may extend outside and along a length of the flexible body 1216.

Medical instrument 1226 can additionally house cables, linkages, or other actuation controls (not shown) that extend between its proximal and distal ends to controllably the bend distal end of medical instrument 1226. Flexible body 1216 can also house cables, linkages, or other steering controls (not shown) that extend between drive unit 1204 and distal end 1218 to controllably bend distal end 1218 as shown, for example, by broken dashed line depictions 1219 of distal end 1218. In some examples, at least four cables are used to provide independent “up-down” steering to control a pitch motion of distal end 1218 and “left-right” steering to control a yaw motion of distal end 1218. In embodiments in which medical instrument system 1200 is actuated by a robotically-assisted assembly, drive unit 1204 can include drive inputs that removably couple to and receive power from drive elements, such as actuators, of the teleoperational assembly. In some embodiments, medical instrument system 1200 can include gripping features, manual actuators, or other components for manually controlling the motion of medical instrument system 1200. The information from tracking system 1230 can be sent to a navigation system 1232 where it is combined with information from visualization system 1231 and/or the preoperatively obtained models to provide the physician or other operator with real-time position information.

This description and the accompanying drawings that illustrate various embodiments should not be taken as limiting. Various mechanical, compositional, structural, electrical, and operational changes may be made without departing from the scope of this description and the invention as claimed, including equivalents. In some instances, well-known structures and techniques have not been shown or described in detail so as not to obscure the disclosure. Like numbers in two or more figures represent the same or similar elements. Furthermore, elements and their associated features that are described in detail with reference to one embodiment may, whenever practical, be included in other embodiments in which they are not specifically shown or described. For example, if an element is described in detail with reference to one embodiment and is not described with reference to another embodiment, the element may nevertheless be claimed as included in the other embodiment.

For the purposes of this specification and appended claims, unless otherwise indicated, all numbers expressing quantities, percentages, or proportions, and other numerical values used in the specification and claims, are to be understood as being modified in all instances by the term “about,” to the extent they are not already so modified. Accordingly, unless indicated to the contrary, the numerical parameters set forth in the following specification and attached claims are approximations that may vary depending upon the desired properties sought to be obtained. At the very least, and not as an attempt to limit the application of the doctrine of equivalents to the scope of the claims, each numerical parameter should at least be construed in light of the number of reported significant digits and by applying ordinary rounding techniques.

It is noted that, as used in this specification and the appended claims, the singular forms “a,” “an,” and “the,” and any singular use of any word, include plural referents unless expressly and unequivocally limited to one referent. As used herein, the term “include” and its grammatical variants are intended to be non-limiting, such that recitation of items in a list is not to the exclusion of other like items that can be substituted or added to the listed items.

Further, this description's terminology is not intended to limit the invention. For example, spatially relative terms—such as “beneath”, “below”, “lower”, “above”, “upper”, “proximal”, “distal”, and the like—may be used to describe one element's or feature's relationship to another element or feature as illustrated in the figures. These spatially relative terms are intended to encompass different positions (i.e., locations) and orientations (i.e., rotational placements) of a device in use or operation in addition to the position and orientation shown in the figures. For example, if a device in the figures is turned over, elements described as “below” or “beneath” other elements or features would then be “above” or “over” the other elements or features. Thus, the illustrative term “below” can encompass both positions and orientations of above and below. A device may be otherwise oriented (rotated 90 degrees or at other orientations) and the spatially relative descriptors used herein interpreted accordingly.

Further modifications and alternative embodiments will be apparent to those of ordinary skill in the art in view of the disclosure herein. For example, the devices and methods may include additional components or steps that were omitted from the diagrams and description for clarity of operation. Accordingly, this description is to be construed as illustrative only and is for the purpose of teaching those skilled in the art the general manner of carrying out the present teachings. It is to be understood that the various embodiments shown and described herein are to be taken as illustrative. Elements and materials, and arrangements of those elements and materials, may be substituted for those illustrated and described herein, parts and processes may be reversed, and certain features of the present teachings may be utilized independently, all as would be apparent to one skilled in the art after having the benefit of the description herein. Changes may be made in the elements described herein without departing from the spirit and scope of the present teachings and following claims.

It is to be understood that the particular examples and embodiments set forth herein are non-limiting, and modifications to structure, dimensions, materials, and methodologies may be made without departing from the scope of the present teachings.

Other embodiments in accordance with the present disclosure will be apparent to those skilled in the art from consideration of the specification and practice of the invention disclosed herein. It is intended that the specification and examples be considered as illustrative and for example only, with the following claims being entitled to their fullest breadth, including equivalents, under the applicable law.

1. A suturing device comprising:
a housing, the housing defining an arcuate needle track, the arcuate needle track having a first end and a second end, wherein the housing further defines a gap between the first end and second end of the arcuate needle track, and further wherein the gap is configured to receive tissue therein to be sutured; and
a drive to advance an arcuate needle about the arcuate needle track and across the gap, the drive comprising at least one drive pawl operably coupled to an actuator, the at least one drive pawl being configured to engage and disengage from the arcuate needle, the drive being configured to advance the at least one drive pawl along a drive pawl pathway from a starting position to an ending position during a drive stroke, wherein the drive pawl pathway is linear when the at least one drive pawl is not engaged with the arcuate needle.

2. The suturing device of claim 1, wherein the drive pawl pathway is linear when the at least one drive pawl is engaged with and advancing the arcuate needle.

3. The suturing device of claim 1, wherein the drive pawl pathway is curved when the at least one drive pawl is engaged with and advancing the arcuate needle.

4. The suturing device of claim 1, wherein the drive pawl pathway forms a tangent with respect to a needle pathway traversed by the arcuate needle.

5. The suturing device of claim 1, wherein the drive pawl pathway is laterally offset with respect to a central longitudinal axis of the suturing device.

6. The suturing device of claim 1, wherein the at least one drive pawl includes a first drive pawl and a second drive pawl, wherein the first drive pawl engages the arcuate needle proximate a first lateral side of the arcuate needle track, and the second drive pawl engages the arcuate needle proximate a second lateral side of the arcuate needle track.

7. The suturing device of claim 6, wherein the first drive pawl and second drive pawl both traverse a linear drive pawl pathway from the starting position to the ending position.

8. The suturing device of claim 7, wherein the first drive pawl and second drive pawl move in tandem when the actuator is actuated.

9. The suturing device of claim 7, wherein the first drive pawl and second drive pawl are mechanically coupled.

10. The suturing device of claim 9, wherein the drive further comprises a yoke comprising a first distally extending arm and a second distally extending arm, and further wherein the first drive pawl is operably coupled to the first distally extending arm and the second drive pawl is operably coupled to the second distally extending arm.

11. The suturing device of claim 9, wherein the first drive pawl is antipodally located with respect to the second drive pawl.

12. The suturing device of claim 9, wherein the first drive pawl and second drive pawl physically contact the arcuate needle simultaneously when the arcuate needle is in a predetermined position within the arcuate needle track.

13. The suturing device of claim 6, wherein the first drive pawl is configured to be actuated independently of the second drive pawl.

14. The suturing device of claim 6, wherein the first drive pawl is configured to advance the arcuate needle along a first rotational direction along the arcuate needle track when the first drive pawl is advanced along a distal direction, and further wherein the second drive pawl is configured to advance the arcuate needle along a second rotational direction opposite the first rotational direction along the arcuate needle track when the second drive pawl is advanced along a distal direction.

15. The suturing device of claim 14, wherein the first drive pawl is configured to advance the arcuate needle along the second rotational direction along the arcuate needle track when the second drive pawl is retracted along a proximal direction.

16-32. (canceled)

33. A suturing device comprising:
a housing, the housing defining a gap between a first housing portion and a second housing portion, wherein the gap is configured to receive tissue therein to be sutured; and
a drive to advance an arcuate needle across the gap during a drive stroke, the arcuate needle having a length of suturing material extending proximally therefrom;
an elongate tool having a detachable coupling at a distal end of the elongate tool, the detachable coupling defining therein at least one needle engagement surface to engage with a tip portion of the arcuate needle, the detachable coupling further defining a cinch to engage a proximal portion of the length of suturing material, wherein engagement of the arcuate needle with the detachable coupling forms a loop of suturing material that can be shortened in length by advancing the length of suturing material along a proximal direction through the cinch.

34. The suturing device of claim 33, wherein the housing comprises an arcuate needle track, the arcuate needle track having a first end and a second end, wherein the gap is defined between the first end and second end of the arcuate needle track.

35-37. (canceled)

38. A suturing device comprising:
a housing, the housing defining an arcuate needle track, the arcuate needle track having a first end and a second end, wherein the housing further defines a gap between the first end and second end of the arcuate needle track, and further wherein the gap is configured to receive a tissue therein to be sutured; and
a drive to advance an arcuate needle about the arcuate needle track and across the gap, the drive comprising a first drive pawl and a second drive pawl;
wherein the first drive pawl is configured to be advanced by the drive along a first direction into contact with the arcuate needle to advance the arcuate needle in a forward direction along the needle track by a first distance, and
wherein the second drive pawl is configured to be retracted by the drive along the first direction into contact with the arcuate needle to continue to advance the arcuate needle in the forward direction along the needle track by a second distance.

39. The suturing device of claim 38, wherein the first drive pawl engages the arcuate needle proximate a first lateral side of the arcuate needle track, and the second drive pawl engages the arcuate needle proximate a second lateral side of the arcuate needle track.

40. The suturing device of claim 38, wherein the first drive pawl is configured to advance the arcuate needle along a first rotational direction along the arcuate needle track when the first drive pawl is advanced along a distal direction, and further wherein the second drive pawl is configured to advance the arcuate needle along the first rotational direction along the arcuate needle track when the second drive pawl is retracted along a proximal direction.

41-60. (canceled)
